(19) United States
(12) Patent Application Publication
(10) Pub. No.: US 2023/0390441 A1
(43) Pub. Date: Dec. 7, 2023

(54) HIGH-SPEED NANO MIST, PRODUCTION METHOD AND PRODUCTION DEVICE, PROCESSING METHOD AND PROCESSING DEVICE, AND MEASUREMENT METHOD AND MEASUREMENT DEVICE FOR SAME

(71) Applicant: TOHOKU UNIVERSITY, Sendai-shi, Miyagi (JP)

(72) Inventors: Takehiko SATO, Sendai-shi (JP); Tomoki NAKAJIMA, Sendai-shi (JP); Yunchen XIAO, Sendai-shi (JP); Shigeru FUJIMURA, Sendai-shi (JP)

(73) Assignee: TOHOKU UNIVERSITY, Sendai-shi, Miyagi (JP)

(21) Appl. No.: 18/033,215

(22) PCT Filed: Oct. 26, 2021
(86) PCT No.: PCT/JP2021/039443

§ 371 Date: Apr. 21, 2023

(30) Foreign Application Priority Data
Oct. 27, 2020 (JP) 2020-179943

(51) Int. Cl. A61L 2/18; B08B 3/02; B05B 7/26; B05B 7/00; B05B 1/00

(52) U.S. Cl. CPC A61L 2/186; B08B 3/026; B05B 1/005; B05B 7/0075; B05B 7/26

(57) ABSTRACT

A high-speed nano mist is a group of liquid droplets having a particle diameter of 1 nm to 10000 nm and flying at a speed of 50 m/s to 1000 m/s.

### TECHNICAL FIELD

The present invention relates to a high-speed nano mist, a production method and production device, a processing method and processing device, and a measurement method and measurement device for the same.

The present application claims priority based on Japanese Patent Application No. 2020-179943 filed on Oct. 27, 2020, and contents thereof are incorporated herein.

### BACKGROUND ART

A cleaning technique using a mixed jet flow of steam and water is developed.

For example, NPL 1 below describes a technique capable of cleaning a particle, a photoresist, or the like on a wafer surface without using a chemical solution by mixing water with steam at a constant pressure and spraying the mixture from a nozzle.

In the technique described in NPL 1, clean steam is generated from pure water by electric heating, and is mixed with ultrapure water at about 100 mL/min to 500 mL/min at a nozzle inlet. Next, NPL 1 describes that a steam pressure is set to about 0.1 MPa to 0.3 MPa at the nozzle inlet, and a desired mixed jet flow can be ejected by ejecting the steam from the nozzle having an opening diameter of 3.8 mm.

As a technique for removing plaque by microscopic liquid droplets, NPL 2 below describes a technique of spraying microscopic liquid droplets from a hand piece having an air nozzle and a water nozzle at a high speed at a pressure of 0.15 MPa. NPL 2 describes a content of researching a relation between microscopic liquid droplets having a size of 10 pm to 70 pm and an ability to remove the plaque according to an ejection speed.

### CITATION LIST

### Non Patent Literature

NPL 1: Toshiyuki Sanada, et al., “Development of cleaning technique using spray mixed with steam and water”, Journal of jet flow engineering Vol. 24, No. 3 (2007) 4-10

NPL 2: Satoshi Uehara et al. Removal Mechanism of Artificial Dental Plaque by Impact of Micro-Droplets, ECS Journal of Solid State Science and Technology, 8(2) N20-N24 (2019)

### SUMMARY OF INVENTION

### Technical Problem

As a result of various studies on a cleaning property of water droplets such as steam used in a cleaning technique, the present inventor finds that a nano-order mist attains an extremely specific effect as compared with a micron-order mist. The present inventor finds that cleaning, sterilization, and surface processing can be performed with a function that is not attained so far by causing the nano-order mist to collide with an object or an object present in an object space at a high speed, and the invention is achieved.

Further, the present inventor finds that the collision of the nano-order high-speed mist described above is excellent in dry, drug free, and water-supersaving effect, which cannot be achieved in the related art, and the invention is achieved.

An object of the invention is to provide a high-speed nano mist, a production method and production device, a processing method and processing device, and a measurement method and measurement device for the same, which can solve the above problem by causing the high-speed nano mist to collide with an object or an object present in an object space.

### Solution to Problem

(1) The high-speed nano mist according to the invention is a group of liquid droplets having a particle diameter of 1 nm to 10000 nm and flying at a speed of 50 m/s to 1000 m/s.
(2) A production method for a high-speed nano mist according to the invention is for producing the high-speed nano mist which is a group of liquid droplets having a particle diameter of 1 nm to 10000 nm and flying at a speed of 50 m/s to 1000 m/s.
(3) The production method for a high-speed nano mist according to the invention preferably includes using water as the high-speed nano mist, and ejecting water vapor from the water contained in a sealed container and a pressurized gas supplied to the sealed container from a jet nozzle provided in the sealed container.
(4) A processing method according to the invention preferably includes performing at least one of sterilization, cleaning, and surface processing in a state in which a usage amount of a liquid is reduced without using a drug in a dried state by producing the high-speed nano mist, which is the group of the liquid droplets having the particle diameter of 1 nm to 10000 nm and flying at the speed of 50 m/s to 1000 m/s, and by causing the high-speed nano mist to collide with a target object.
(5) The processing method according to the invention preferably further includes using water as the high-speed nano mist, and ejecting water vapor from the water contained in a sealed container and a pressurized gas supplied to the sealed container from a jet nozzle provided in the sealed container.
(6) In the processing method according to the invention, it is preferable that a phenomenon is used in which OH radical or hydrogen peroxide is generated at a time of producing the high-speed nano mist.
(7) In a measurement method for a high-speed nano mist according to the invention, a phenomenon in which a current flows or a phenomenon in which a voltage changes at a collision surface of a conductor to which the high-speed nano mist is sprayed is used by producing the high-speed nano mist and spraying the high-speed nano mist to the conductor. The high-speed nano mist is a group of liquid droplets having a particle diameter of 1 nm to 10000 nm and flying at a speed of 50 m/s to 1000 m/s.
(8) A production device for a high-speed nano mist according to the invention is for producing the high-speed nano mist, which is a group of liquid droplets having a particle diameter of 1 nm to 10000 nm and flying at a speed of 50 m/s to 1000 m/s, and causing the high-speed nano mist to collide with a target object.
(9) The production device for a high-speed nano mist according to the invention includes: a sealed container configured to use water as the high-speed nano mist and to contain the water; a gas supply source configured to supply a pressurized gas to the sealed container; and a jet nozzle configured to eject the water vapor from the water and the pressurized gas supplied to the sealed container.
(10) A processing device according to the invention is preferably for performing at least one of the sterilization, the cleaning, and the surface processing in a state in which a usage amount of a liquid is reduced without using a drug in a dried state by producing a high-speed speed nano mist, which is a group of liquid droplets having a particle diameter of 1 nm to 10000 nm and flying at a speed of 50 m/s to 1000 m/s, and by causing the high-speed nano mist to collide with a target object.
(11) The processing device according to the invention preferably includes: a sealed container configured to use water as the high-speed nano mist and to contain the water; a gas supply source configured to supply a pressurized gas to the sealed container; and a jet nozzle configured to eject water vapor from the water and the pressurized gas supplied to the sealed container.
(12) A measurement device for a high-speed nano mist according to the invention is for measuring a current flowing or a voltage generated on a collision surface of a conductor to which the high-speed nano mist is sprayed by producing the high-speed nano mist and spraying the high-speed nano mist to the conductor. The high-speed nano mist is a group of liquid droplets having a particle diameter of 1 nm to 10000 nm and flying at a speed of 50 m/s to 1000 m/s.

### Advantageous Effects of Invention

According to the high-speed nano mist and the production method for the same in the invention, the steam generated inside the sealed container by a pressure exceeding 1 atm applied to a liquid contained in the sealed container and a steam pressure of the liquid can be ejected from the jet nozzle as the high-speed nano mist at the high speed. Unlike a general mist mainly containing liquid droplets of a micron order or larger sizes, the high-speed nano mist has a unique cleaning property and sterilizing property, and can perform various types of processing such as cleaning, sterilizing, and surface processing on a sprayed space or a surface of a sprayed target object in a finally dried state.

Therefore, it is suitable for removal or sterilization of a biofilm of a bacterium or the like that cannot be easily cleaned by a general cleaning method in the related art based on a perforation effect of the high-speed nano mist, and a virus can be easily inactivated by spraying the high-speed nano mist to a virus or the like.

Since the high-speed nano mist ejected from the jet nozzle is extremely small liquid droplets, the usage amount of the liquid can be reduced, and water-supersaving type cleaning, sterilization, and surface processing can be performed. Therefore, it is possible to perform various types of processing such as the cleaning, the sterilization, and the surface processing with a small amount of liquid if the high-speed nano mist is used for a long time.

### BRIEF DESCRIPTION OF DRAWINGS

FIG. 1 is a configuration diagram showing a nano mist production device according to a first embodiment of the invention.

FIG. 2 is a perspective view showing an example of a jet nozzle applied to the nano mist production device.

FIG. 3 is a side view showing an example of the jet nozzle.

FIG. 4 is a front view showing an example of the jet nozzle.

FIG. 5 is an explanatory view showing an example of a case in which the nano mist production device shown in FIG. 1 is used for hand cleaning.

FIG. 6 is an explanatory view showing an example of a case in which the nano mist production device shown in FIG. 1 is used for a dry shower.

FIG. 7 is an explanatory view showing an example of a case in which the nano mist production device shown in FIG. 1 is used for a dry curtain.

FIG. 8 is an explanatory view showing an example of a case in which the nano mist production device shown in FIG. 1 is used for instrument sterilization.

FIG. 9 is an explanatory view showing an example of a case in which the nano mist production device shown in FIG. 1 is used for body cleaning.

FIG. 10 is an explanatory view showing an example of a case in which the nano mist production device shown in FIG. 1 is used for food sterilization.

FIG. 11 is an explanatory view showing an example of a case in which the nano mist production device shown in FIG. 1 is used for substrate cleaning.

FIG. 12 is an explanatory view showing an example of a case in which the nano mist production device shown in FIG. 1 is used for domestic animal cleaning.

FIG. 13 is a perspective view of a built-in heater.

FIG. 14 is a configuration diagram in which a gas supply pipe, a heater, and a heat insulation material are removed from a nano mist production device according to a second embodiment of the invention.

FIG. 15 is a configuration diagram of the nano mist production device according to the second embodiment of the invention.

FIG. 16 is a photograph showing a state in which a jet flow of a high-speed nano mist ejected using the nano mist production device shown in FIG. 1 is irradiated with a green laser and is visualized.

FIG. 17 is a diagram showing a result of measuring speed distribution, which relates to a nano mist produced by the nano mist production device shown in FIG. 1.

FIG. 18 is a graph showing a relation between a pressure and a current that flows when an aluminum plate is irradiated with the nano mist produced by the nano mist production device shown in FIG. 1.

FIG. 19 is a graph showing a relation between a separation distance from the jet nozzle to the aluminum plate and the current that flows when the aluminum plate is irradiated with the high-speed nano mist as shown in FIG. 18.

FIG. 20 is a graph showing a result of measuring a sampled nano mist using an electron spin resonance device (ESR device) and detecting OH radical, which relates to the nano mist produced by the nano mist production device shown in FIG. 1.

FIG. 21 is a laser microscope photograph showing a surface state of an organic film prepared for observing an effect of the nano mist produced by the nano mist production device shown in FIG. 1.

FIG. 22 is a laser microscope photograph showing a surface state after irradiating the organic film for 5 sec with the nano mist produced by the nano mist production device shown in FIG. 1.

FIG. 23 is an enlarged photograph showing an example of a state of imaging a state in which the nano mist produced by the nano mist production device shown in FIG. 1 is caused to collide with a front surface side of a transparent substrate at a high speed by an ICCD camera from a back surface side of the transparent substrate.

FIG. 24 is a 3D display setting diagram showing an example of a result of observation with a laser microscope, which relates to the surface state of the organic film obtained by irradiating the organic film with the nano mist produced by the nano mist production device shown in FIG. 1.

FIG. 25 is a partially enlarged view of a region having two minute holes (dark portions) on the surface of the organic film observed by the laser microscope.

FIG. 26 is an analysis diagram showing a result of measuring depths of the minute holes (dark portions) in the observation result obtained using the laser microscope shown in FIG. 25.

FIG. 27 is a microscope photograph (SEM: 10 kV, 2000 times) showing a biofilm of Staphylococcus aureus attached on an artificial blood vessel.

FIG. 28 is a microscope photograph (SEM: 10 kV, 2000 times) showing a state after irradiating a biofilm equivalent to the biofilm shown in FIG. 27 for 5 sec with the high-speed nano mist produced by the nano mist production device shown in FIG. 1.

FIG. 29 is a microscope photograph (SEM: 10 kV, 9000 times) showing a state after irradiating a biofilm composed of Staphylococcus aureus formed on a stainless substrate with oxygen gas at 4 atm for 5 sec.

FIG. 30 is a microscope photograph (SEM: 10 kV, 9000 times) showing a state after irradiating the biofilm composed of Staphylococcus aureus formed on the stainless substrate with the high-speed nano mist produced by the nano mist production device shown in FIG. 1 for 5 sec.

FIG. 31 is a photograph showing an example of a result of performing a cleaning test using a commercially available cleaning indicator using the high-speed nano mist produced by the nano mist production device shown in FIG. 1.

FIG. 32 is a diagram showing a voltage change when the nano mist production device shown in FIG. 1 produces the high-speed nano mist.

FIG. 33 is a diagram showing a voltage change when the high-speed nano mist is produced by changing a heating temperature of the jet nozzle in the nano mist production device shown in FIG. 15.

FIG. 34 is a diagram showing arrangement of a measurement device that measures temperature distribution of the high-speed nano mist.

FIG. 35 is a diagram showing a relation between a temperature and a position of the high-speed nano mist.

FIG. 36 is a diagram showing a relation between the pressure and the position of the high-speed nano mist.

(a) of FIG. 37 is a schlieren image of a gas and (b) of FIG. 37 is a schlieren image of a water vapor mixed gas.

FIG. 38 is a diagram showing a relation between a current that flows when the aluminum plate is irradiated with the high-speed nano mist and the separation distance between the jet nozzle and the aluminum plate.

FIG. 39 is a diagram showing a relation between a potential of the aluminum plate and time when the aluminum plate is irradiated with the high-speed nano mist.

FIG. 40 is a diagram showing a relation between the amount of hydrogen peroxide produced and a sampling time period.

### DESCRIPTION OF EMBODIMENTS

### First Embodiment

Hereinafter, an example of embodiments of the invention will be described in detail with reference to the accompanying drawings. In the drawings used in the following description, in order to make features easier to understand, the features may be enlarged and shown for convenience.

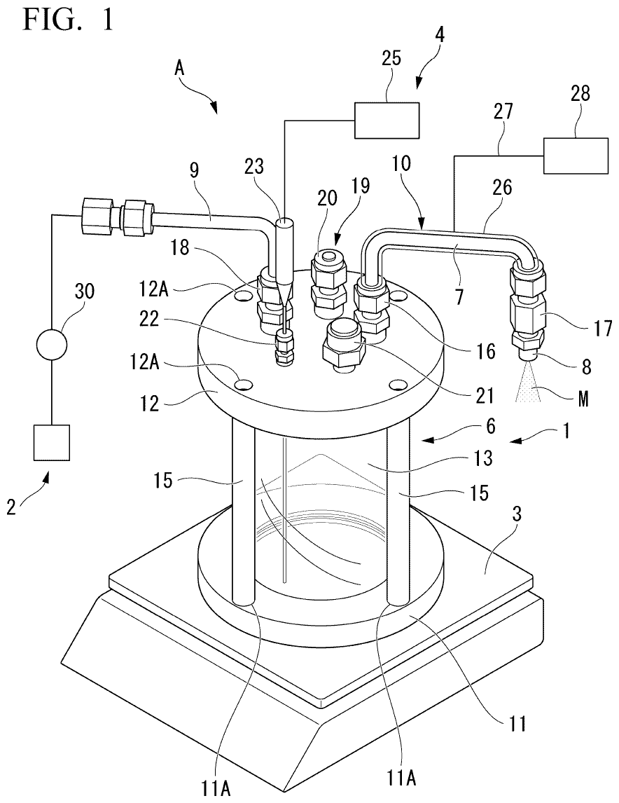

FIG. 1 shows a nano mist production device according to a first embodiment of the invention, and a nano mist production device A according to the embodiment mainly includes a nano mist production device main body 1, a gas supply source 2 connected to the nano mist production device main body 1, a heating device 3, and a temperature measuring device 4. The gas supply source 2 supplies a pressurized gas to a sealed container 6. The nano mist production device main body 1 includes the sealed container 6 that contains a liquid (for example, water), a jet nozzle 8 connected to the sealed container 6 via a jet pipe 7, a gas supply pipe 9 that connects the gas supply source 2 to the sealed container 6, and a nozzle heater 10 disposed around the jet pipe 7.

The sealed container 6 includes a disk-shaped bottom plate 11 constituting a bottom wall, a disk-shaped top plate 12 constituting a ceiling wall, a cylindrical wall body 13 constituting a peripheral wall, and a plurality of (four in an example in FIG. 1) strut members 15 provided between the bottom plate 11 and the top plate 12.

As an example, the bottom plate 11, the top plate 12, and the strut members 15 are made of a metal such as stainless steel such as SUS 316 specified by JIS. Outer diameters of the bottom plate 11 and the top plate 12 are about 110 mm, the wall body 13 has a cylindrical shape and is made of quartz glass or the stainless steel, and the sealed container 6 is formed in a cylindrical shape having a height of about 150 mm as a whole.

Four counterbore portions 11A are formed at an equal interval in a circumferential direction near an outer peripheral edge portion of an upper surface of the bottom plate 11, and four counterbore portions 12A are formed at an equal interval in a circumferential direction near an outer peripheral edge portion of a lower surface of the top plate 12. The bottom plate 11 and the top plate 12 are disposed in parallel to each other such that these counterbore portions 11A and 12A face each other in the upper-lower direction, and the strut members 15 are provided between the top counterbore portions 11A and the bottom counterbore portions 12A. Screw holes are formed at both end portions of the strut member 15, and the bottom plate 11, the top plate 12, and the strut member 15 are coupled to one another by screwing a coupling bolt not shown into the screw hole of the strut member 15 via the counterbore portion 11A or the counterbore portion 12A, and the sealed container 6 is implemented.

A concave portion not shown into which a bottom portion side of the wall body 13 can be inserted is formed on an upper surface side of the bottom plate 11, a bottom portion of the wall body 13 is inserted into the concave portion, and a sealing material such as an 0-ring is fitted to the periphery of the bottom portion, whereby the bottom portion of the wall body 13 is airtightly joined to the bottom plate 11.

A concave portion not shown into which a top portion side of the wall body 13 can be inserted is formed on a lower surface side of the top plate 12, a top portion of the wall body 13 is inserted into the concave portion, and a sealing material such as an 0-ring is fitted to the periphery of the top portion, whereby the top portion of the wall body 13 is airtightly joined to the top plate 12.

Five insertion holes are formed on an upper surface side of the top plate 12, and these insertion holes are open in the sealed container 6. Among the five insertion holes, the jet pipe 7 is connected to an opening of a first insertion hole via a cylindrical joint member 16, extends horizontally to outside of the top plate 12, and is bent downward on a side of the top plate 12, and the jet nozzle 8 is attached to a distal end side of the jet pipe 7 in a downward direction via a cylindrical joint member 17.

The gas supply pipe 9 is joined to an opening of a second insertion hole through a cylindrical joint member 18. A cylindrical joint member 19 is connected to an opening of a third insertion hole, and a sealing nut 20 is detachably attached to an upper portion of the joint member 19. By detaching the sealing nut 20, the joint member 19 serves as an inlet of a liquid such as water.

A safety valve 21 is attached to an opening of a fourth insertion hole. The safety valve 21 operates at a predetermined pressure such as 0.5 MPa, and is provided such that an internal pressure of the sealed container 6 does not increase more than necessary.

A joint member 22 for attaching a thermometer is attached to an opening of a fifth insertion hole, a temperature sensor 23 is inserted into the sealed container 6 via the joint member 22, an internal temperature of the sealed container 6 measured by the temperature sensor 23 is measured, and the temperature can be displayed on a display device 25. The temperature sensor 23 has, for example, a distal end portion inserted deeply into the sealed container 6 to measure a temperature of the liquid contained in the sealed container 6. The temperature sensor 23 and the display device 25 constitute the temperature measuring device 4. As the temperature sensor 23, for example, a K type thermocouple may be used.

A heater not shown is attached to the jet pipe 7 in a manner of extending from a joint portion with the joint member 16 to an outer peripheral portion of the jet nozzle 8, a heat insulation material 26 is wound in a manner of covering the jet pipe 7 and the heater, and the nozzle heater 10 is implemented. FIG. 1 briefly shows the nozzle heater 10. A wiring 27 for energization of the heater is drawn out to outside of the heat insulation material 26, and the jet pipe 7 can be heated by the nozzle heater 10 by connecting an attachment plug 28 connected to the wiring 27 to a commercial power source or the like if necessary. It is desired that when the jet pipe 7 is heated by the nozzle heater 10, the jet pipe 7 can be heated to about a boiling point of the liquid contained in the sealed container 6.

The gas supply pipe 9 is connected to the gas supply source 2 such as a gas cylinder or a compressor, and a pressure gauge 30 is incorporated in the gas supply pipe 9. Therefore, a gas such as air can be supplied from the gas supply source 2 to the inside of the sealed container 6 at a desired pressure. In addition to the air, the gas supply source 2 may supply an inert gas such as nitrogen gas. The gas to be supplied is not limited to the air and the inert gas.

The sealed container 6 is provided on the heating device 3 such as a hot plate. Therefore, the inside of the sealed container 6 can be heated by operating the heating device 3, and the liquid such as the water contained in the sealed container 6 can be heated to a target temperature to generate steam.

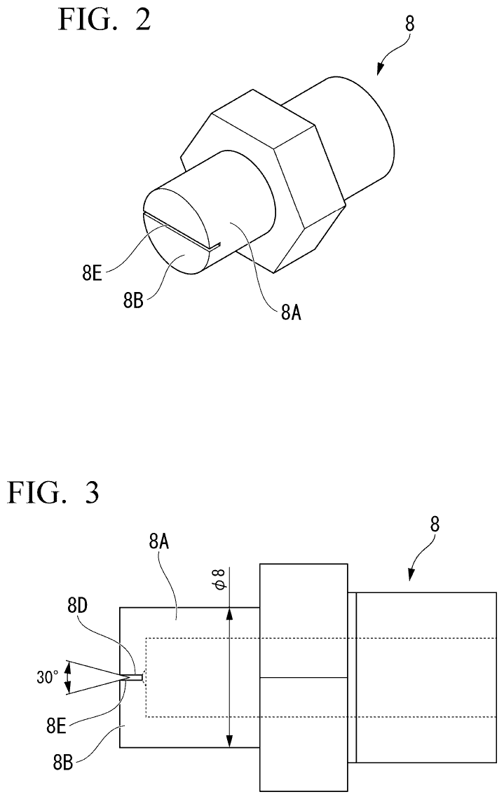

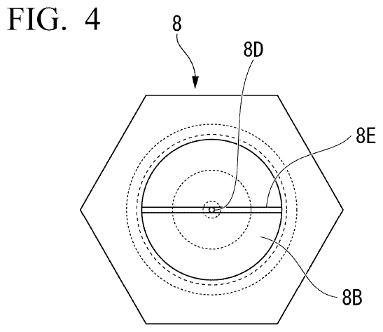

The jet nozzle 8 ejects water vapor generated from the water contained in the sealed container 6 and the pressurized gas supplied to the sealed container 6. In the jet nozzle 8, for example, as shown in FIGS. 2 to 4, a distal end wall 8B is formed at a distal end of a tubular portion 8A, and a nozzle hole 8D is formed at a central portion of the distal end wall 8B. A V-shaped groove 8E with a concave slit passing through the central portion of a front wall is formed on a front surface side of the distal end wall 8B, and the nozzle hole 8D is open on the bottom surface side of the central portion in a length direction of the slit. An inner diameter of the nozzle hole 8D may be, for example, about 0.1 mm to 2.0 mm.

A shape and the inner diameter of the jet nozzle 8 are not particularly limited, and the V-shaped groove 8E may have any shape such as a recessed groove or a parallel groove. The nozzle may be a jet nozzle having no V-shaped groove 8E, or may be a nozzle having any structure, such as a diffuser type nozzle or a concentric type nozzle.

A method of producing the high-speed nano mist using the nano mist production device A implemented as described above and colliding an object with the high-speed nano mist will be described. Here, the high-speed nano mist is a group of liquid droplets having a particle diameter of 1 nm to 10000 nm and flying at a speed of 50 m/s to 1000 m/s. In the present embodiment, in a production method for the high-speed nano mist, the high-speed nano mist which is the group of the liquid droplets having the particle diameter of 1 nm to 10000 nm and flying at the speed of 50 m/s to 1000 m/s is produced using the nano mist production device A. For example, the water is used as the high-speed nano mist, and the water vapor generated from the water contained in the sealed container 6 and the pressurized gas supplied to the sealed container 6 are ejected from the jet nozzle 8 provided in the sealed container 6. The production device of the high-speed nano mist produces a high-speed nano mist M and causes the high-speed nano mist M to collide with a target object. Hereinafter, the method for colliding the object with the high-speed nano mist will be described.

The nano mist production device A is assembled as shown in FIG. 1, and the gas supply pipe 9 is connected to the gas supply source 2. The temperature sensor 23 is connected to the sealed container 6, the sealing nut 20 is removed from the joint member 18, and a required amount of water is injected into the sealed container from an inlet of the joint member 18. When the water is injected into the sealed container 6, the water is injected into the sealed container 6 such that the sealed container has a little residual space, instead of injecting the water such that the sealed container 6 is full of the water. For example, the water is injected such that about a few centimeters of the residual space is left. Alternatively, a gas is ejected into the water. At this time, heating of the gas can be promoted by ejecting the gas as fine bubbles.

After a predetermined amount of water is injected, the sealing nut 20 is closed to seal the sealed container 6. Thereafter, the water is heated by the heating device 3, and the jet pipe 7 is heated by the heater. The gas such as air is supplied from the gas supply source 2 to the residual space of the sealed container 6, and the residual space is adjusted to be under a gas pressure exceeding 1 atm. For example, the gas pressure is adjusted to a range of about 2 atm to 10 atm, and more preferably about 2 atm to 5 atm.

Although pressure resistance of the sealed container 6 to be applied is not limited, it is desired that a sealing structure or the like of the sealed container 6 is not larger than necessary, and is preferably about 2 atm to 5 atm in order not to be restricted by a regulation of a high-pressure container. When a size of the sealed container 6 is increased and an airtight structure is a more precise structure, a device having a pressure of about 6 atm to 12 atm may be used.

As an example of a case of using the sealed container 6, a water temperature is preferably set to be a boiling state of about 152° C. at 5 atm. At 5 atm, the water is boiled at about 152° C. The pressure in the sealed container 6 is further higher by 1 atm with respect to a gauge pressure indicated by the pressure gauge 30 shown in FIG. 1. Therefore, for example, when the gauge pressure of the pressure gauge 30 indicates 4 atm, the inside of the sealed container 6 has an absolute pressure of about 5 atm, and in this case, the water is boiled at about 152° C.

When a nano mist is generated and ejected as the high-speed nano mist, it is desired to heat the nano mist such that the temperature is close to the boiling point of the water contained in the sealed container 6, and when an ejection pressure may be somewhat low, the temperature may be a temperature lower by about 10% to 20% than the boiling point, for example, about 120° C. to 150° C. in a case of the above 5 atm. The boiling point of the water is about 100° C. at 1 atm, about 121° C. at 2 atm, about 134° C. at 3 atm, and about 144° C. at 4 atm, and thus the water temperature corresponding to each gas pressure can be adopted. The temperature of the residual space of the sealed container 6 affects a condensing state of water molecules evaporated from the liquid water. It is desired to reduce aggregation of the water molecules as much as possible by setting the temperature of the residual space to a temperature equal to or higher than the boiling point, and the condensation may be advanced at a temperature equal to or lower than the boiling point temperature to change the particle diameter of the water droplets contained in the high-speed nano mist M. A water vapor generation amount may be changed by lowering the water temperature from the boiling point when the high-speed nano mist M is produced to reduce the number of the liquid droplets of the mist.

For example, when the gas pressure is adjusted to 2 atm or more at the absolute pressure and the temperature is close to that of boiling water, the high-speed nano mist M can be ejected from the jet nozzle 8. In the sealed container 6, the steam is discharged from the water into the residual space, the steam is condensed by pressurized air to become the high-speed nano mist M mainly containing nano-order fine liquid droplets, and the high-speed nano mist M is ejected from the jet nozzle 8 at a high speed as it is. It is considered that the nano mist is produced even at 2 atm, but the ejection speed of the nano mist is low. Therefore, when the nano mist is ejected at the high speed, a pressure range of 3.5 atm or more at the absolute pressure, for example, a range of 3.5 atm to 12 atm, more preferably about 3.5 atm to 10 atm is desired.

In general, when the gas is sealed in the sealed container and a nozzle diameter is sufficiently small, if a gas pressure difference is 3 atm or more, the gas can be ejected from the nozzle at a speed close to a sonic speed. Therefore, in order to eject the nano mist at the high speed in the sealed container 6, the gas pressure difference is desired to be large. In the present application, since the nano mist produced in the residual space of the sealed container 6 is partially condensed when the nano mist is ejected from the jet nozzle 8, it is considered that the nano mist can be ejected at the high speed as it is by applying a higher pressure, unlike a general non-condensable gas. Therefore, it is desired to use the gas pressure described above.

The high-speed nano mist M also includes ejection of a part of micron-order liquid droplets, and when the nano mist is ejected from the sealed container 6 at the above pressure, it is possible to produce the high-speed nano mist M as a steam jet flow mainly containing the nano-order mist. When white light is applied to a front space of the jet nozzle 8, a steam jet flow mainly containing the micron-order liquid droplets becomes a steam jet flow that can be checked by a naked eye such that the jet flow of the steam presents a white color. However, the high-speed nano mist M, which is the steam jet flow mainly containing the nano-order mist, becomes a steam jet flow that cannot be checked with the naked eye even when the white light is applied to a space on a distal end side of the jet nozzle 8. The high-speed nano mist M mainly containing the nano-order mist can be visualized by applying a green laser (wavelength: 532 nm) to the space on the distal end side of the jet nozzle 8. It is considered that, when the mist containing a large amount of nano-order mist and containing a part of the micron-order mist is the high-speed nano mist M which mainly contains a mist of about several pm as the micron-order mist and which additionally contains a large amount of nano-order mist, the mist can be visualized by the irradiation with the green laser as described above.

Therefore, the high-speed nano mist M mainly containing the nano-order mist can be referred to as the steam jet flow that cannot be checked with the naked eye in the state in which the ejection from the distal end of the jet nozzle 8 is irradiated with the white light but can be visually recognized when irradiated with laser light.

It is considered that the nano-order liquid droplets described above mainly contain liquid droplets having the particle diameter of 10000 nm or less, more preferably 1000 nm or less, and about 1 nm to 10000 nm, more preferably about 1 nm to 1000 nm as an example when reference is made within a scope. It is difficult to directly confirm presence of the high-speed liquid droplets having such a particle diameter range, but it can be confirmed from various test results to be described later that the nano mist production device A having the above configuration can jet the mist mainly containing the nano mist at the high speed.

As can be confirmed from the test results to be described later, the above high-speed nano mist M is ejected from the jet nozzle 8 at a speed of about 20 m/s to 1000 m/s, and the main high-speed nano mist is ejected from the ejection nozzle 8 at a speed of about 50 m/s to 300 m/s.

When 200 mL of water is contained in the sealed container 6 under the above condition, and the high-speed nano mist M is ejected under the above condition, the high-speed nano mist M can be ejected continuously for about 1 hour to 2 hours depending on a diameter of the jet nozzle 8.

Since a pressure of the sum of a pressure of, for example, 2 atm to 12 atm supplied from the gas supply source 2 and a steam pressure of the water generated when the water becomes steam inside the sealed container 6 acts on the inside of the sealed container 6, the high-speed nano mist M can be ejected from the jet nozzle 8.

The high-speed nano mist M has various features. As an example, excellent detergency and excellent sterilization ability are attained, and an excellent surface processing effect is exhibited. Since the liquid droplets having the particle diameter of about 1 nm to 10000 nm have a small particle diameter, the liquid droplets are instantaneously dried and evaporated when sprayed onto a cleaning portion of an object for cleaning, and thus the cleaning portion can be finally cleaned without being wetted. The high-speed nano mist M is sprayed onto an object to be sterilized, the object to be sterilized can be sterilized without wetting a portion to be finally sterilized. An effect of being capable of cleaning and sterilizing a site to which the high-speed nano mist M is sprayed and being in a drying state after cleaning and sterilizing has been demonstrated by a biofilm removal test to be described later.

The nano mist having the particle diameter of about 1 nm to 1000 nm is instantaneously dried and evaporated when the nano mist is collided with the object, and thus cleaning and sterilization can be performed without finally wetting a collision site of the liquid droplets as described above. On the other hand, when a large number of liquid droplets having a particle diameter of 1 μm to 10 μm or more are contained, the drying time of the liquid droplets becomes long, and as a result, the cleaning site or the sterilization site is wetted.

For example, if the biofilm of a bacterium is attached to a blood vessel or the like, the biofilm can be easily removed by spraying the high-speed nano mist M for about several seconds. The biofilm is a biofilm composed of the bacterium such as Staphylococcus aureus, and in general, even the biofilm cannot be easily removed by spraying cleaning water or oxygen, the biofilm can be removed by spraying the high-speed nano mist M for about several seconds.

Although a reason for this is unclear in detail, it may be related to a fact that presence of OH radical has been detected in a high-speed nano mist sampling test to be described later.

It is considered that, as a result of the collision with the nano mist ejected at the high speed, the biofilm, which is difficult to be removed by a method such as simply spraying with air, is penetrated by the nano-order liquid droplets like a bullet, the bacterium is pierced and destroyed, and the removal of the biofilm can be implemented in several seconds.

When the cleaning and the sterilization by the collision of the high-speed nano mist M are performed, the biofilm can be removed by spraying for several seconds. Therefore, for example, when a surgical site after surgery and surroundings thereof are cleaned and sterilized, the cleaning and sterilization can be performed in a shorter time by spraying the high-speed nano mist M. The spraying can be performed for about 1 hour to 2 hours with 200 mL of water as described above, and thus even when the high-speed nano mist M is sprayed onto a wide area for cleaning and sterilization, the cleaning and sterilization can be performed with a small amount of water. That is, water-supersaving type cleaning and sterilization can be performed. When the above water-supersaving type cleaning and sterilization is used as the surface processing, water-supersaving type surface processing can be performed.

In terms of injection time of the water, the water can be continuously ejected for a long period of time when capacity of the sealed container 6 to be used is increased. Therefore, the above injection time is merely an example.

In the above description, when the water is injected into the sealed container 6 shown in FIG. 1, the water is injected such that about a few centimeters of the residual space is left. Alternatively, the water may be injected to be in a fully charged state without leaving the residual space, and the gas may be supplied into the sealed container 6 from the gas supply pipe 9. A distal end of the gas supply pipe 9 may be drawn into the sealed container 6, and the gas may be injected into the sealed container 6 with bubbling.

In any case, it is effective if the nano mist having the particle diameter of about 1 nm to 10000 nm can be ejected at the high speed of about 50 m/s to 1000 m/s from the distal end of the jet nozzle 8 to cause the nano mist to collide with the target object.

It is desired that the high-speed nano mist M is ejected from the jet nozzle 8 in a state in which the water contained in the sealed container 6 is boiled. Alternatively, the high-speed nano mist M may be generated while maintaining a temperature slightly lower than the boiling point, and may be ejected from the jet nozzle 8.

The above high-speed nano mist M can be applied to cleaning, sterilization, and surface processing in various situations. In the processing method and processing device according to the present disclosure, at least one of the sterilization, the cleaning, and the surface processing is performed in a state in which a usage amount of the liquid is reduced without using a drug in a dried state by producing the high-speed nano mist and causing the high-speed nano mist to collide with the target object. Specifically, the water is used as the high-speed nano mist, and the water vapor generated from the water contained in the sealed container 6 and the pressurized gas supplied to the sealed container 6 are ejected from the jet nozzle 8 provided in the sealed container 6 to perform the processing. In the processing method, it is preferable to use a phenomenon in which OH radical or hydrogen peroxide is produced at the time of producing the high-speed nano mist.

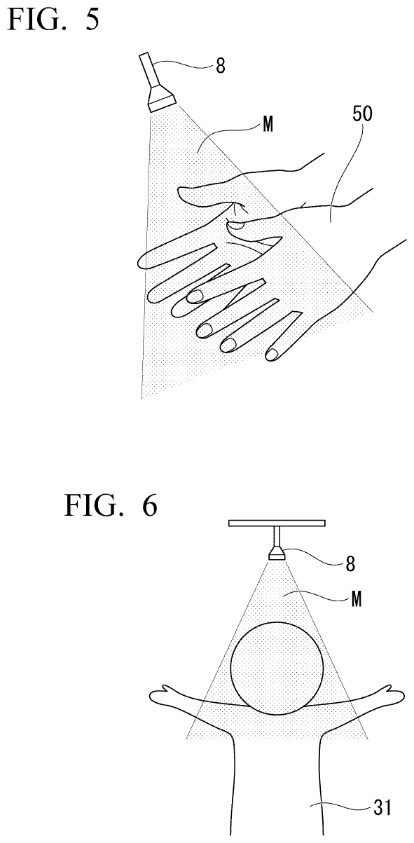

For example, as shown in FIG. 5, by placing a hand (object) 50 of a user below the jet nozzle 8, the high-speed nano mist M can be sprayed onto the hand 50, and a water-supersaving type dry sterilizing and hand cleaning operation can be implemented.

In a case of the sealed container 6 described above, the high-speed nano mist can be sprayed for 1 hour with 200 mL of water, and thus when a size of the sealed container 6 is increased, the hand cleaning can be performed for a longer continuous time.

This means that, for example, it is possible to easily and reliably perform the water-supersaving type hand cleaning in a desert region, a barren, or the like, in which it is not easy to obtain the water. It is possible to reduce infrastructure development related to water supply and sewerage in a region where water is precious, and it is possible to attain a remarkable effect in the region where water is precious.

When the jet nozzle 8 is applied as a shower as shown in FIG. 6, the above high-speed nano mist M can be used as a water-supersaving dry shower for cleaning a human body (object) 31. For example, in an evacuation facility such as a disaster site, when cleaning using the above high-speed nano mist M is performed, it is possible to contribute to implementation of saving water, implementation of the hand cleaning and cleaning in an environment in which a water supply is stopped, simplification of the hand cleaning, simplification of a bath, simplification of washing, and the like.

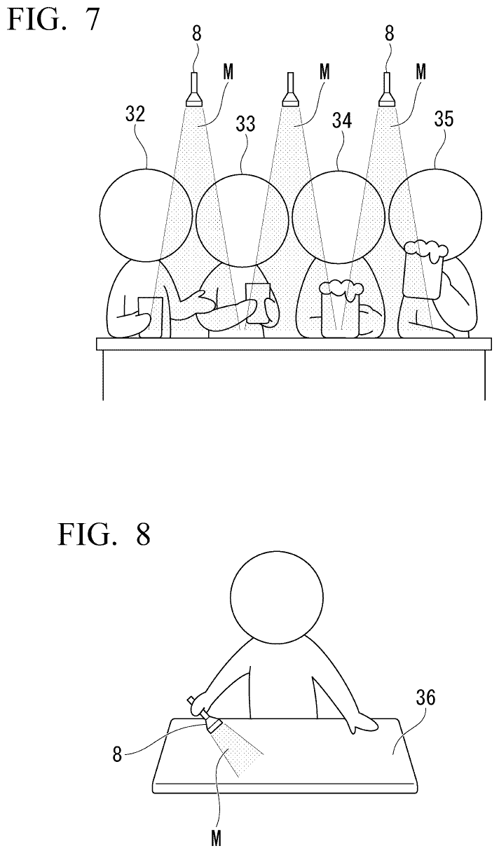

The above high-speed nano mist M is excellent in sterilization effect, and thus in a restaurant or the like, as shown in FIG. 7, when a plurality of eaters or drinkers 32, 33, 34, and 35 eat and drink close to each other on left and right sides, the high-speed nano mist M can be applied in place of an acrylic board currently used for separating the eaters or drinkers. For example, by downward providing the jet nozzle 8 above a space (object space) between the eaters or drinkers 32, 33, 34, and 35, the high-speed nano mist M can be sprayed downward like a shower and a curtain of the high-speed nano mist M can be generated. If an object such as a bacterium or a virus is present in the space between the eaters or drinkers, the object can be hit with the high-speed nano mist M and destroyed, or can be inactivated. The high-speed nano mist M can be used as a dry curtain instead of an acrylic plate in the related art by a curtain of the high-speed nano mist M ejected downward from the jet nozzle 8. The high-speed nano mist M can be used as the dry curtain, and thus the high-speed nano mist M can be continuously used for a long period of time without wetting a sprayed space.

It is said that the virus that causes infection are floated in the air as an aerosol in a state of adhering to particles such as small water droplets and particles such as dust. It is said that a human is infected with the virus by suctioning the floated aerosol. In particular, in a place of eating and drinking, in a site in which people are crowded, the aerosol containing the virus is likely to be generated along with cough or conversation.

By spraying the above high-speed nano mist M to the aerosol (object), the virus can be inactivated and rendered harmless. When the above high-speed nano mist is sprayed onto the bacterium or the like, it has been confirmed that the bacteria can be destroyed by destroying a cell membrane or a cell wall of the bacterium in a test to be described later, and thus the high-speed nano mist M is particularly effective in a case in which the bacterium, the virus, or the like is destroyed and rendered harmless. Therefore, in the restaurant or the site in which people are crowded, there is an effect that it is possible to eat and drink in a so-called closed space and crowded place and close-contact setting (3Cs) state, or to eat and drink and have a conversation with security when people are gathered. In the case of the sealed container 6 described above, the nano mist can be sprayed for about 1 hour to 2 hours with 200 mL of water, and thus when the size of the sealed container 6 is increased, continuous long-time injection of the high-speed nano mist can be performed according to a business hour of the restaurant. Needless to say, the place in which the sterilization and the cleaning are performed using the high-speed nano mist M is not limited to the restaurant, and may be a place in which people are likely to be crowded, for example, a concert hall or a theater, a gathering place, a live house, a hospital, a house, and a space in a building.

It is considered that the speed of the high-speed nano mist M also decreases when a position is away from the jet nozzle 8, and an effect of lowering the virus and the bacterium floating in the space can be attained by adsorbing and colliding with the virus and the bacterium. Therefore, in addition to the above effect of destroying the bacterium and the virus, the object such as the bacteria and the viruses floating in the space can be lowered to a floor or ground, and an effect of moving the object to a position in which the bacterium and the virus are not suctioned into the human body can be attained. For example, the bacterium and the virus can be inactivated by dropping onto the floor or the ground.

As shown in FIG. 8, the above high-speed nano mist M is also effective for cleaning a cooking tool (object) 36 such as a chopping board, and can clean and sterilize the cooking tool 36 by spraying the high-speed nano mist M toward the cooking tool 36 through the jet nozzle 8. When the cleaning and the sterilization are performed, a portion to be cleaned and a portion to be sterilized can be maintained in the dry state.

Since there are various types of cooking tools in an eating and drinking establishment and the like, the high-speed nano mist M can be widely used for cleaning a general cooking tool. Accordingly, it is possible to sterilize and remove a drug resistant bacterium and a bacterium causing food poisoning to reduce occurrence of the food poisoning in the eating and drinking establishment.

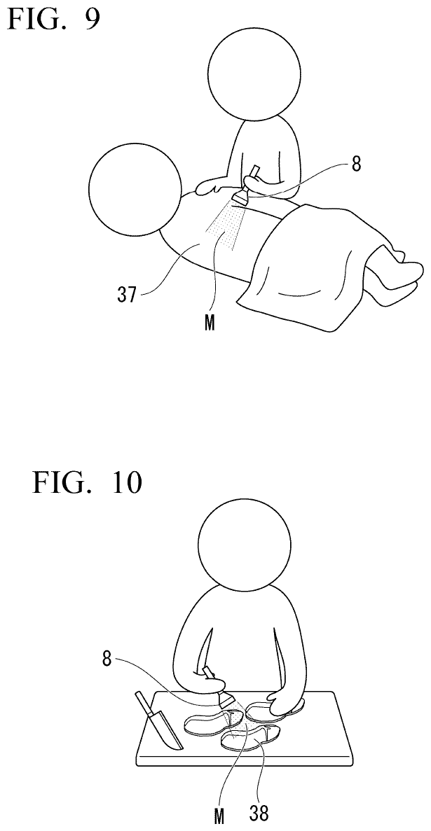

As shown in FIG. 9, when the above high-speed nano mist M is applied to a human body (object) 37 such as a bedridden person as a shower at a care site, the high-speed nano mist M can be used as a water-supersaving type dry shower for cleaning and sterilizing the human body 37 using the jet nozzle 8. In this application, since the cleaning and the sterilization can be performed while the dry state is maintained, it is possible to clean and sterilize the human body 37 such as the bedridden person without wetting the human body. Therefore, it is possible to eliminate manpower shortage of bathing assistance work in a storage facility of a bedridden person and the like.

As shown in FIG. 10, the above high-speed nano mist M is also effective for cleaning a foodstuff (object) 38 such as meat, and dry-cleaning and dry-sterilization can be performed on the foodstuff 38 by spraying the high-speed nano mist M toward the foodstuff 38 through the jet nozzle 8. When the cleaning and the sterilization are performed, a portion to be cleaned and a portion to be sterilized can be maintained in the dry state. Therefore, the cleaning and the sterilization can be performed without affecting flavor of the foodstuff 38 and the like.

Since the high-speed nano mist M can sterilize an agricultural product with no pesticide, and thus can be effectively used for the sterilization of the agricultural product. In this case, it is possible to reduce a disease of the agricultural product due to the bacterium and the virus without damaging the agricultural product using the high-speed nano mist M for sterilizing a non-agrichemical vegetable.

The high-speed nano mist M can also be applied to an oral care application by spraying the high-speed nano mist M to an object such as a tooth neck portion, a gingiva portion, and the like of a person or an animal.

As shown in FIG. 11, the above high-speed nano mist M can be used for cleaning and surface processing of a semiconductor substrate 39 by spraying the high-speed nano mist M ejected from the jet nozzle 8 onto the semiconductor substrate (object) 39.

Currently, in a semiconductor factory, switching from a wet process to a dry process is advanced in a memory manufacturing process and the like, but in the semiconductor manufacturing process, there is a problem that the usage amount of cleaning water in a substrate cleaning process is extremely large. A structure of a semiconductor such as a memory is complicated, several hundred layers are stacked on a semiconductor wafer, and a large number of wirings and contact holes are processed in each layer. Therefore, in some memories, it is said that 1.7 trillions of holes may be formed on the semiconductor wafer.

A cleaning step of some semiconductor wafers is said to have 350 steps to 4000 steps, a step of using the cleaning water is essential in removal of an organic substance, removal of an oxide film, removal of an ion, alcohol replacement, and the like, and it is said that the cleaning water whose amount is equivalent to the amount usually used by a small town is used in some large factories.

When a part of the cleaning step and the surface processing step is switched to the cleaning and surface processing with the high-speed nano mist M described above, there is an effect that a large amount of water can be saved in the substrate cleaning step and the surface processing step, and high-speed cleaning work and surface processing work can be implemented.

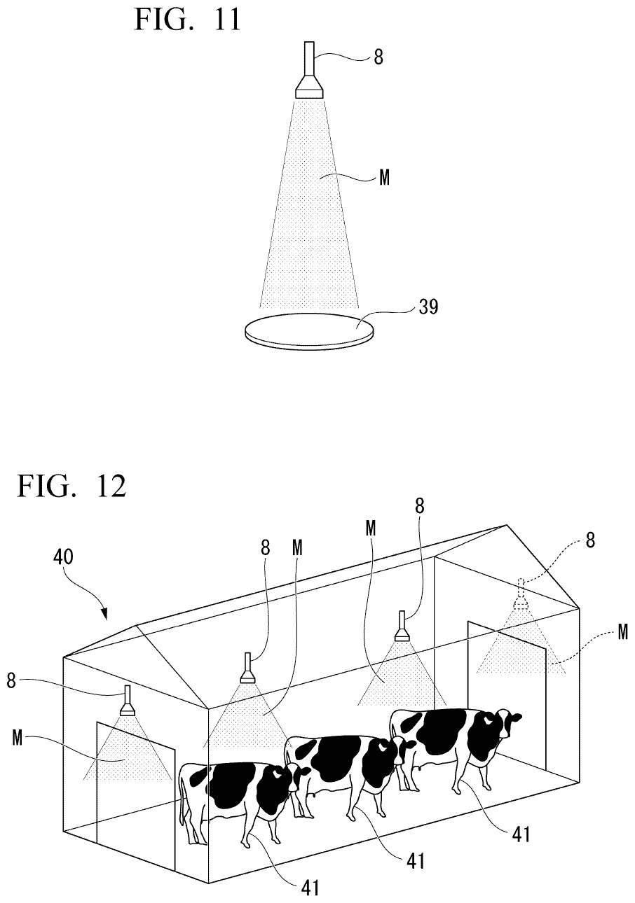

As shown in FIG. 12, the above high-speed nano mist M can be applied to cleaning and sterilization of a domestic animal as a dry shower using the jet nozzle 8.

For example, the sealed container 6 is provided above cows (objects) 41 in a cowshed 40, and the high-speed nano mist M is constantly sprayed from the jet nozzle 8, so that the cow 41 can be constantly sterilized and constantly cleaned. When the jet nozzle 8 is formed above an inlet and an outlet of a livestock and the high-speed nano mist M is ejected downward to an object space, it is possible to perform hygiene management such that the bacterium and the virus are not brought into the livestock from outside. As a placement position of the jet nozzle 8, it is desired that the vicinity of the inlet and the vicinity of the outlet of the cowshed 40 are provided, and it is desired that the jet nozzle 8 is provided in and around a portion which may be a main body as an intrusion path of the bacterium and the virus.

As described above, when the cows 41 are constantly sterilized and constantly cleaned, it is possible to eliminate a risk that the cows are infected with a panic disease.

The above high-speed nano mist M can be used for constant sterilization, constant cleaning, and constant disinfection in a domestic animal facility such as a pig farm, a bird rearing and egg-laying facility, or the like. Accordingly, it is possible to improve cleanness of a domestic animal rearing environment, and the high-speed nano mist M can be effectively utilized to infection prevention of a domestic animal contagious disease, such as prevention of a bird influenza, prevention of a classical swine fever, and a prevention of foot-and-mouth disease.

The above high-speed nano mist M is composed of the water droplets. Therefore, the high-speed nano mist M is harmless, can be carried out without adversely affecting the domestic animals, and can be provided at a low cost because the high-speed nano mist M is not a drug. By using the above high-speed nano mist M, it is possible to sterilize a necessary site and a necessary space in a harmless state for the domestic animal without using a disinfectant as the drug.

In the example described above, the high-speed nano mist M is produced from the water. However, the liquid used for producing the high-speed nano mist is not limited to the water, and may be a liquid other than the water containing a disinfectant, a cleaning liquid, and other necessary components.

In the above example, an example has been described in which one of the cleaning, the sterilization, and the surface processing is performed, and the above high-speed nano mist production device A may be widely applied to general processing for other purposes using the water or a liquid other than the water described above.

### Second Embodiment

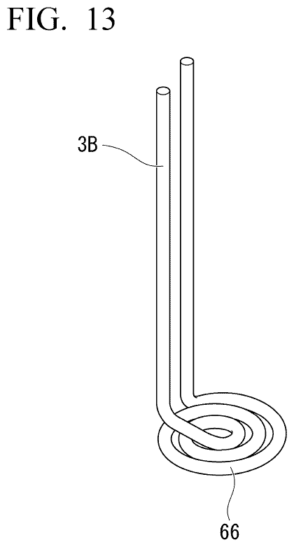

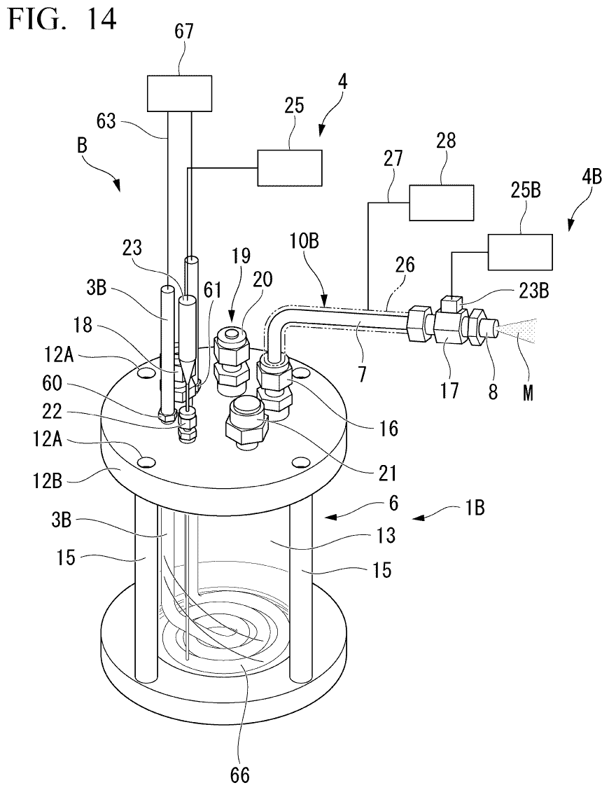

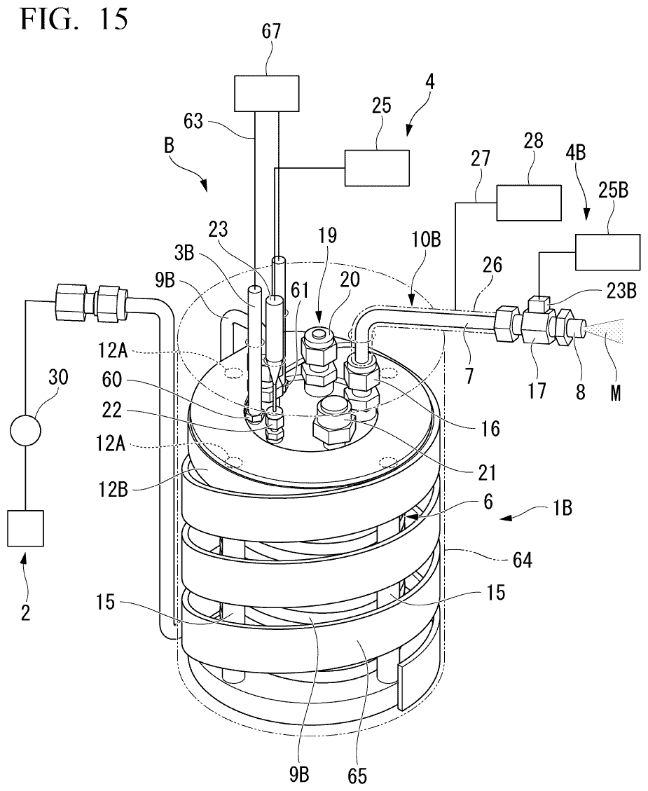

FIG. 13 is a perspective view of a built-in heater 3B. FIGS. 14 and 15 show a nano mist production device according to a second embodiment of the invention. For the description, FIG. 14 shows a configuration of a nano mist production device in which a gas supply pipe 9B, a heater 65, and a heat insulation material 64 are removed. FIG. 15 shows a nano mist production device according to the second embodiment to which the gas supply pipe 9B, the heater 65, and the heat insulation material 64 are attached. A nano mist production device B according to the second embodiment mainly includes a nano mist production device main body 1B, the gas supply source 2 connected to the nano mist production device main body 1B, the built-in heater 3B, the temperature measuring device 4, and a nozzle side temperature measuring device 4B. The nano mist production device main body 1B includes the sealed container 6 that contains a liquid, the jet nozzle 8 connected to the sealed container 6 via the jet pipe 7, the gas supply pipe 9B that connects the gas supply source 2 to the sealed container 6, and a nozzle heater 10B disposed around the jet pipe 7. Hereinafter, only contents different from components of the nano mist production device A will be described for components of the nano mist production device B according to the second embodiment, and detailed description of contents common to the components of the nano mist production device A may be omitted.

Seven insertion holes are formed on an upper surface side of a top plate 12B, and these insertion holes are open in the sealed container 6. Among the seven insertion holes, the jet pipe 7 is connected to an opening of a first insertion hole via the cylindrical joint member 16, the jet pipe 7 extends horizontally to outside of the top plate 12, and the jet nozzle 8 is attached to the distal end side of the jet pipe 7 via the cylindrical joint member 17.

The gas supply pipe 9B is joined to an opening of a second insertion hole through the cylindrical joint member 18. The cylindrical joint member 19 is connected to an opening of a third insertion hole, and the sealing nut 20 is detachably attached to an upper portion of the joint member 19. By detaching the sealing nut 20, the joint member 19 serves as an inlet of a liquid such as water.

The safety valve 21 is attached to an opening of a fourth insertion hole. The safety valve 21 operates at a predetermined pressure such as 0.5 MPa, and is provided such that an internal pressure of the sealed container 6 does not increase more than necessary.

The joint member 22 for attaching a thermometer is attached to an opening of a fifth insertion hole, the temperature sensor 23 is inserted into the sealed container 6 via the joint member 22, an internal temperature of the sealed container 6 measured by the temperature sensor 23 is measured, and the temperature can be displayed on the display device 25. The temperature sensor 23 has, for example, a distal end portion inserted deeply into the sealed container 6 to measure a temperature of the liquid contained in the sealed container 6. The temperature sensor 23 and the display device 25 constitute the temperature measuring device 4. As the temperature sensor 23, for example, a K type thermocouple may be used.

A joint member 60 for attaching the built-in heater 3B is attached to an opening of a sixth insertion hole, and a joint member 61 for attaching the built-in heater 3B is attached to an opening of a seventh insertion hole. The built-in heater 3B is disposed inside the sealed container 6 via the joint members 60 and 61. A wiring 63 for energization of the built-in heater is drawn out to outside of the heat insulation material 64, and the inside of the sealed container can be heated by the built-in heater 3B by connecting an attachment plug 67 connected to the wiring 63 to a commercial power source or the like. By using the built-in heater 3B, the water contained in the sealed container 6 can be heated more efficiently than a case in which the heater is disposed outside. Accordingly, ejection of condensed water can be reduced. The built-in heater 3B may heat only a portion (a spiral portion 66 in FIG. 9) disposed on a bottom surface side of the sealed container 6. By heating in this manner, the water can be effectively used.

A heater not shown is attached to the jet pipe 7 in a manner of extending from a joint portion with the joint member 16 to an outer peripheral portion of the jet nozzle 8, the heat insulation material 26 is wound in a manner of covering the jet pipe 7 and the heater, and the nozzle heater 10B is implemented. A temperature sensor 23B for measuring a temperature of a nozzle is provided in the vicinity of the jet nozzle 8. The temperature sensor 23B and a display device 25B constitute the nozzle side temperature measuring device 4B. As the temperature sensor 23B, for example, a K type thermocouple may be used.

FIGS. 14 and 15 briefly show the nozzle heater 10B. The wiring 27 for energization of the heater is drawn out to outside of the heat insulation material 26, and the jet pipe 7 can be heated by the nozzle heater 10 by connecting the attachment plug 28 connected to the wiring 27 to the commercial power source or the like if necessary. It is desired that, when the jet pipe 7 is heated by the nozzle heater 10, the jet pipe 7 can be heated to about a boiling point of the liquid contained in the sealed container 6.

The gas supply pipe 9B is connected to the gas supply source 2 such as a gas cylinder or a compressor, and the pressure gauge 30 is incorporated in the gas supply pipe 9B. Therefore, a gas such as air can be supplied from the gas supply source 2 to the inside of the sealed container 6 at a desired pressure. The gas supply pipe 9B is wound along an outer periphery of the wall body 13. The heater 65 is disposed around outside of the gas supply pipe 9B. The gas supply pipe 9B is disposed on the outer periphery of the wall body 13 and the gas supply pipe 9B is heated by the heater 65, so that the gas can be heated before entering an inner container. Accordingly, the ejection of the condensed water can be reduced. In addition to the air, the gas supply source 2 may supply an inert gas such as nitrogen gas. The gas to be supplied is not limited to the air and the inert gas.

The heater 65 covers periphery of the top plate 12B and the gas supply pipe 9B. Since the heater 65 heats the top plate 12B and the gas supply pipe 9B, a frequency of the condensed water can be reduced. The heater 65 is, for example, a ribbon heater capable of performing heating to 400° C. A temperature of the heater 65 is preferably higher than the temperature of the boiling water (for example, in a case of 5 atm (absolute pressure), about 152° C.), and at about 180° C., the condensation amount is reduced. When the temperature of the heater 65 is higher, it is possible to further reduce the condensation of the high-speed nano mist M. In the present embodiment, the heater 65 and the nozzle heater 10B are separately attached, and the heater may be one as long as the heater can heat a target portion.

The heat insulation material 64 covers the heater 65 and the sealed container 6. As described above, since the heat insulation material 64 covers the sealed container 6, it is possible to greatly reduce the generation of the condensed water.

Since a temperature (the temperature measured by the nozzle side temperature measurement device 4B) of a nozzle is changed, a condensation amount of the high-speed nano mist M can be adjusted. In order to detect the amount of water in the sealed container 6, the temperature sensor 23 is inserted, and a water temperature inside the sealed container 6 is measured. After the water temperature reaches, for example, about 152° C. (a boiling point in a case of 5 atm), when the water temperature is changed by ±4° C. or more, heating of the heater is stopped. When the water is reduced and a temperature measurement position is exposed to a gas from the water, the temperature measurement position hits the preheated gas, and the temperature is equal to or higher than the boiling point. Alternatively, when a preheating temperature of the gas is low, the temperature conversely decreases. Therefore, it is found that the water in the sealed container 6 has become equal to or less than a specified value when the water is changed by ±4 degrees or more.

In the measurement method for the nano mist according to the present disclosure, a phenomenon in which a current flows or a phenomenon in which the voltage changes in a collision surface of a conductor on which the high-speed nano mist M is sprayed is used by producing the high-speed nano mist M and spraying the high-speed nano mist M to the conductor. The measurement device according to the present disclosure includes, for example, the nano mist production device A, the conductor not shown, and a power supply not shown. The conductor is, for example, an aluminum plate. In a state in which the power source is connected to the aluminum plate and the other electrode of the power source is grounded, the high-speed nano mist M is sprayed from the nano mist production device A. Since the nano mist is charged, the current flows. By measuring the current, the state of the high-speed nano mist M can be measured. Alternatively, the state of the high-speed nano mist M can be measured by measuring the voltage generated when the high-speed nano mist is sprayed.

### EXAMPLES

### Example 1

The sealed container 6 having the structure shown in FIGS. 1 and 2 was prepared. The bottom plate 11, the top plate 12, and the strut members 15 were formed of SUS 316 specified by JIS. The bottom plate 11 having an outer diameter of 110 mm and a thickness of 12 mm and the top plate 12 having an outer diameter of 110 mm and a thickness of 15 mm are prepared, the wall body 13 is implemented by a cylindrical body made of quartz glass, and the bottom plate 11, the top plate 12, and the wall body 13 were combined to constitute the cylindrical sealed container 6 having a total height of 150 mm. The jet nozzle is made of SUS 316 specified by JIS. A circular concave portion having a depth of 7 mm was formed on an upper surface side of the bottom plate 11 and a lower surface side of the top plate 12, a bottom portion and a top portion of the wall body 13 were fitted into the concave portion via an O-ring, the strut members were aligned with counterbore portions of the bottom plate 11 and the top plate 12, and the strut members were bolted and assembled in a cylinder shape, whereby the sealed container 6 was assembled. In the jet nozzle 8, the tubular portion 8A had a diameter of φ8 mm, and the jet nozzle 8 is used in which a water path having a diameter of φ4.5 mm was formed in the tubular portion 8A and the nozzle hole 8D having a diameter of φ0.7 mm was formed in a central portion of a distal end wall B. The size of the sealed container described above is a size that does not require registration of the pressure container, and is merely used as an example.

The sealed container 6 was provided on a hot plate serving as a heating device. The gas supply pipe 9 was attached to the sealed container 6 and connected to the gas supply source 2 implemented by a gas cylinder, the temperature sensor 23 was connected to the sealed container 6, the sealing nut 20 was removed from the joint member 18, and 200 mL of water was injected into the sealed container from an inlet of the joint member 18. The water is injected such that a residual space having a height of about 2 cm was left in the sealed container 6.

After the water was injected, the sealing nut 20 was closed to seal the sealed container 6. Thereafter, the water was heated in the heating device 3, and the jet pipe 7 was heated to a boiling point or higher by a heater (wire heater CRX-1, manufactured by TOKYO KAGAKU KENKYUSHO CO., LTD.). Air was supplied from the gas supply source 2 to the residual space of the sealed container 6, a gas pressure in the residual space was gradually increased at regular intervals to adjust the gauge pressure to 1 atm to 4.8 atm (2 atm to 5.8 atm as the absolute pressure in the sealed container), and the sealed container 6 was heated by the hot plate to heat the water in the sealed container to a boiling temperature.

The steam jet flow can be jetted from a distal end of the jet nozzle 8 by the above operation. However, the present inventor estimated that the high-speed nano mist mainly containing liquid droplets having a particle diameter of 1 nm to 10000 nm is formed in the sealed container 6 at a gauge pressure of 2.5 atm (absolute pressure: 3.5 atm) or more.

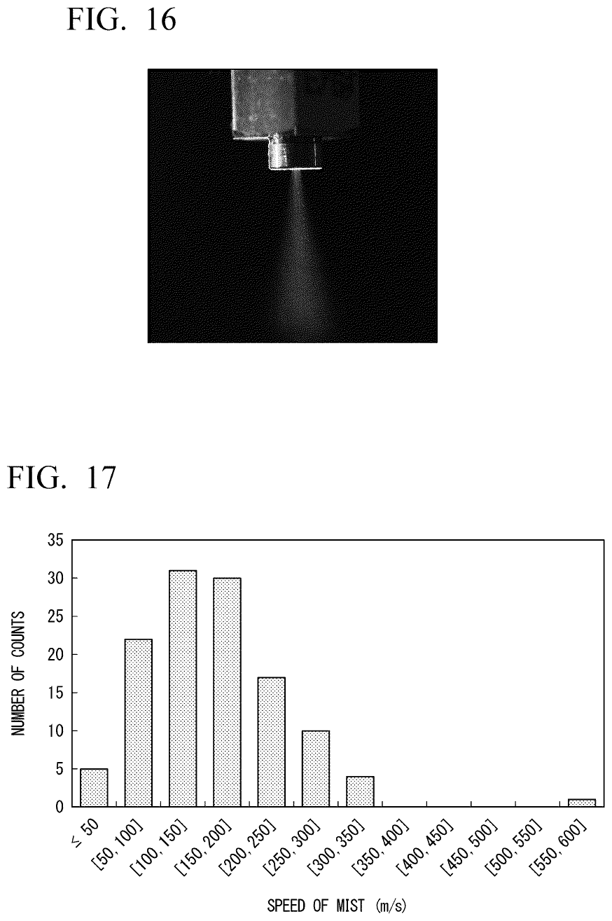

In terms of the pressure of the air to be sent to the residual space, the jet flow of the high-speed nano mist jetted when the gauge pressure was fixed to 4 atm (absolute pressure: 5 atm) was not visually recognized with a naked eye under white illumination light of the environment in which an experiment was performed. Therefore, when a green laser (central wavelength: 532 nm) was emitted toward a region in which the high-speed nano mist was ejected, presence of a steam jet flow (high-speed nano mist) mainly containing the nano mist was imaged by a charge-coupled device (CCD) camera with an image intensifier (ICCD camera) as shown in a photograph in FIG. 16, and the presence thereof was confirmed.

In terms of the high-speed nano mist, high-speed imaging using the ICCD camera was applied to a microscope observation image, and ejection speed distribution of a partial mist of the micron order contained in the high-speed nano mist M was measured within a range of a depth of field of the microscope. When background light which is a laser is incident, and the mist is allowed to pass through and is captured by a high-speed camera at 10 Mfps, the micron-order mist can be seen within the range of the depth of field of the microscope, and thus a speed of the micron-order mist can be measured based on time and a distance at which the mist moves. A result thereof is shown in FIG. 17.

In a graph shown in FIG. 17, a horizontal axis indicates an ejection speed range, and a vertical axis indicates the number of counts of measured mists. For example, [50, 100] on the horizontal axis indicates that 22 counts of mists indicating the ejection speed in a range of m/s to 100 m/s are observed.

In the above measurement method, the micron-order mist can be measured, and it is considered that the nano-order mist is also ejected at the same speed as the mist of the micron-order size.

As shown in the graph in FIG. 17, liquid droplets that can be observed by the microscope are distributed in a range of 20 m/s to 600 m/s, and a main liquid droplet speed is distributed in a range of 50 m/s to 350 m/s. Therefore, it is determined that the nano mist having a smaller particle diameter was also distributed in the range of 20 m/s to 600 m/s in speed, and that the main liquid droplet speed was distributed in the range of 50 m/s to 350 m/s.

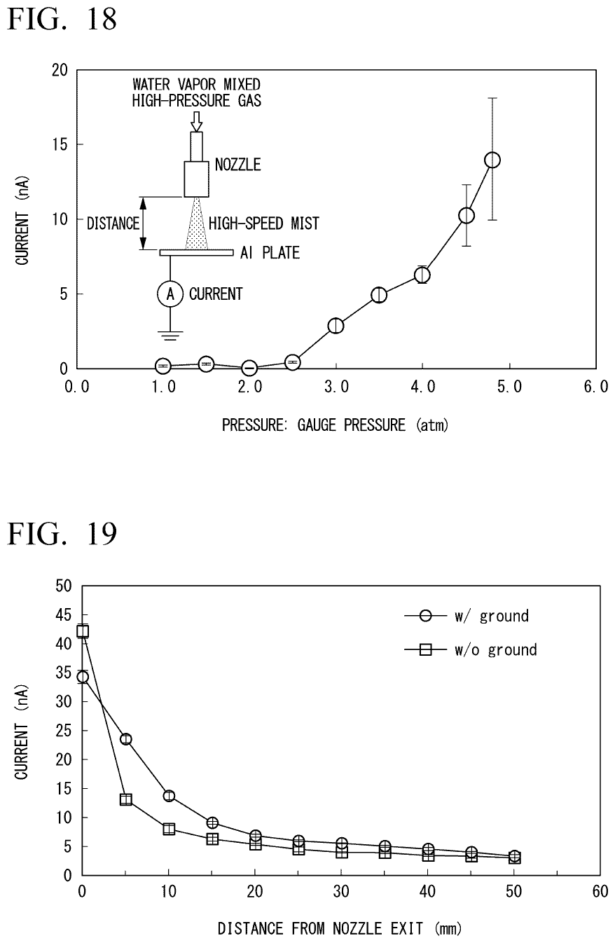

FIG. 18 is an analysis diagram in a case in which a test is performed in which the steam jet flow is ejected downward while gradually increasing a gauge pressure of the air to be sent to the sealed container 6 to 1 atm to 4.8 atm, the aluminum plate is horizontally provided below the jet nozzle 8, and the steam jet flow is sprayed onto the aluminum plate. A power source was connected to a lower surface of the aluminum plate, and the other electrode of the power source was grounded.

As a result, when the gauge pressure of the air to be sent to the sealed container was gradually increased to 1 atm to 4.8 atm, almost no current flows at the gauge pressure of 1 atm to 2.5 atm, the current starts to flow through the aluminum plate when the gauge pressure exceeded 2.5 atm, and the current value increased until the pressure reached 2.5 atm to 4.8 atm (absolute pressure: 3.5 atm to 5.8 atm).

When the gauge pressure to be sent to the sealed container is 4 atm (absolute pressure: 5 atm), the water is boiled at about 152° C.

A reason why the current flows is unclear, and in an atmospheric pressure range exceeding the gauge pressure of 2.0 atm (absolute pressure: 3.0 atm), the steam jet flow is considered to be a jet flow of the high-speed nano mist mainly containing the nano-order liquid droplets.

When the pressure of the air to be applied to the sealed container was set to 4 atm, and continuous ejection of the high-speed nano mist was performed using the above jet nozzle, the amount of water used was 200 mL per hour. In a case of general hand cleaning using water, it is said that 6 L of water is used in 30 sec when the water is continuously ejected from a water supply, and thus the amount of the water used in the same time can be reduced to one several thousandths when the above high-speed nano mist is used for the hand cleaning.

FIG. 19 shows a correlation between a distance from the aluminum plate to the jet nozzle 8 and a flowing current value based on a current measurement result obtained when the pressure of the air sent to the sealed container in the test shown in FIG. 18 is fixed to the gauge pressure of 4 atm (absolute pressure: 5 atm) and the distance from the jet nozzle to the aluminum plate is changed.

W/ground in FIG. 19 means that the sealed container is grounded, and W/O ground means that the sealed container is not grounded.

In a case in which the nano mist is ejected from the jet nozzle, when it is estimated that the nano mist is charged, it is considered that a current flows at a short distance at which a large number of nano mists collide.

FIG. 20 shows a result of sampling the high-speed nano mist generated at the absolute pressure of 2 atm in the sealed container and analyzing the high-speed nano mist by an electron spin resonance device (ESR device). The analysis can be performed by spraying high-speed nano mists into a beaker containing a disodium terephthalate solution (NaTA solution, concentration: 100 mM) for 20 minutes and analyzing the fluorescence spectrum (center wavelength: 425 nm) of 2-hydroxyterephthalic acid (HTA).

When OH radical is present in a disodium terephthalate solution, the OH radical reacts with terephthalic acid to generate the 2-hydroxyterephthalic acid.

When excitation light having a wavelength of 310 nm is incident on the generated 2-hydroxyterephthalic acid, fluorescence having a wavelength of 425 nm is emitted. Using the principle, a calibration curve is created using a standard substance of HTA for quantification, and an absolute amount can be estimated by comparison with the calibration curve. In the analysis, the above NaTA solution having a high concentration was used, and the analysis was performed using the NaTA solution having the concentration such as 0.2 μM, 0.5 μM, and 1 μM as a standard solution.

The analysis was performed under a measurement condition corresponding to an integrated time of 20 sec for a fluorescence spectrum of HTA of the high-speed nano mist, smoothing: 3, and an integrated time of 10 sec for the NaTA solution having the concentration such as 0.2 μM, 0.5 μM, and 1 μM as the standard solution, smoothing: 5. In the experiment, the solution is sampled to measure a fluorescence intensity by a simple spectrometer with lapse of discharge time.

As shown in FIG. 20, although the OH radical has an extremely small amount around a measurement limit, the presence of the OH radical has been detected. Since the amount is minute, it is difficult to estimate the absolute amount. In a graph shown in FIG. 20, a horizontal axis indicates an intensity of an applied magnetic field, and a vertical axis indicates a signal intensity (any unit).

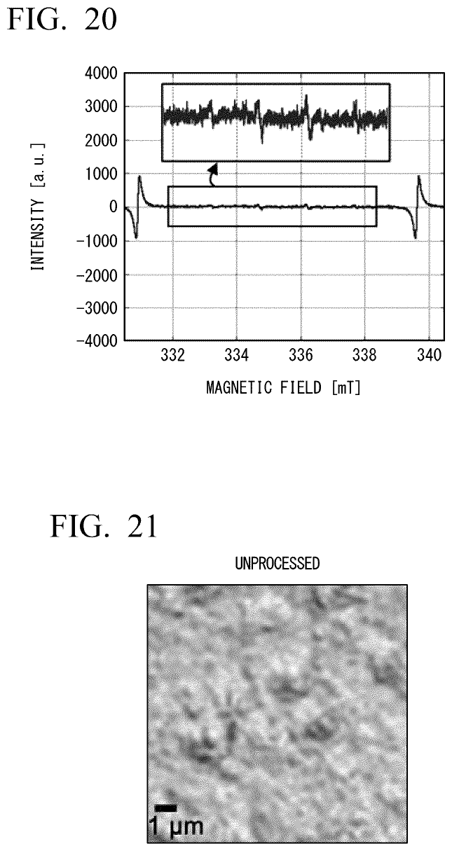

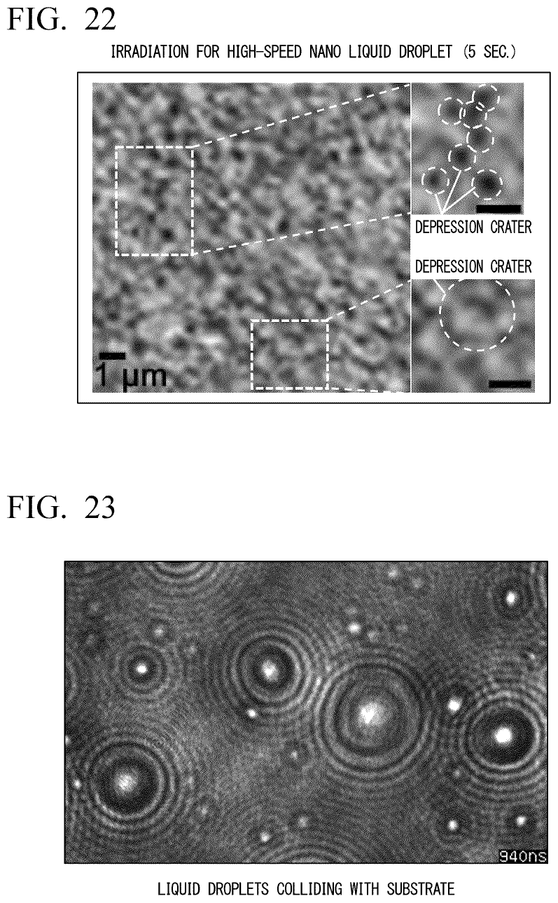

FIG. 21 shows a microscope photograph of an organic film formed on a glass substrate, and FIG. 22 shows a laser microscope photograph of the organic film shown in FIG. 21 after the high-speed nano mist generated by sending the air to the sealed container at the gauge pressure of 4 atm (absolute pressure: 5 atm) is sprayed for 5 sec at a distance of 4 cm from the organic film.

As shown in a photograph shown in FIG. 22, it has been confirmed that a large number of depressions (dark portions) of about 500 nm or less are present in the organic film. When water droplets are sprayed onto the organic film at a high speed to form a depression, a size of the water droplets colliding with the organic film is considered to be smaller than a fraction of the depression, for example, about ⅓ of the depression. It is because it is clear that the water droplets collide with the organic film and spread in a circular shape and the depression having a predetermined radius and depth is formed in a part of the organic film, and the depression is formed by the collision of the water droplets smaller than the inner diameter of the depression.

Therefore, it can be estimated that the water droplets which form a crater-shape depression of about 500 nm shown in FIG. 22 are water droplets having a particle diameter of 300 nm or less. In view of these results, it was estimated that a large number of collisions of the water droplets having the smaller particle diameter occurred on the organic film, and a test was performed as follows.

FIG. 23 shows a result of imaging at the high speed a state in which the pressure of the air sent to the sealed container is fixed to the gauge pressure of 4 atm (absolute pressure: 5 atm), in which the distance between the jet nozzle and the glass substrate is fixed to 4 cm, in which the ICCD camera is provided on a back surface side of the glass substrate, and in which a large number of mists containing the high-speed nano mist collide with the surface of the glass substrate.

Concentric circular ripples having various sizes shown in FIG. 23 show a state in which the water droplets spread in the circular shape as a result of the collision of the water droplets on the glass substrate at the high speed.

In the photograph shown in FIG. 23, a ripple having a size smaller than the size that can be visually recognized in FIG. 23 is not shown up. However, when an original moving image of the photograph is magnified and observed, it is possible to observe an appearance in which countless smaller concentric circular ripples collide with the glass substrate and the concentric circular ripples are generated and disappear.

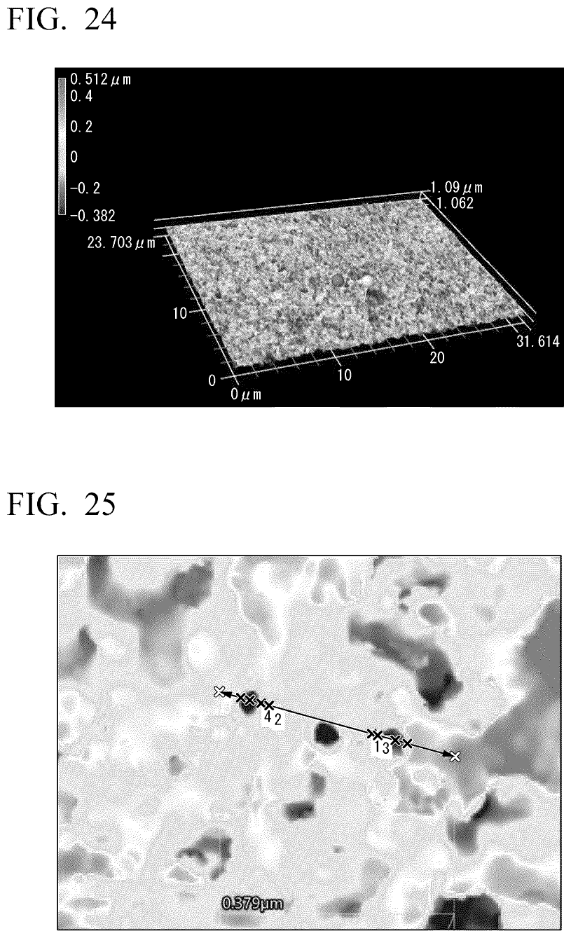

FIGS. 24 to 26 are diagrams illustrating an example of an analysis result of a laser microscope (VK-X1000, manufactured by Keyence Corporation) for a sample sprayed with the high-speed nano mist onto the organic film described above.

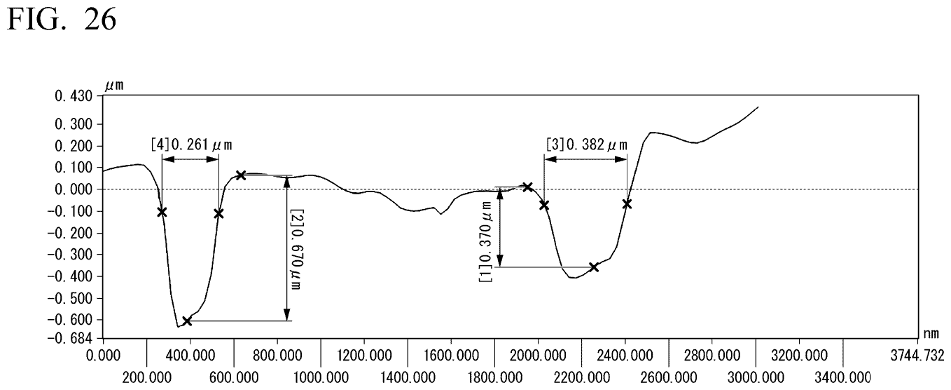

FIG. 24 shows a 3D display setting result, FIG. 25 shows a partial enlarged view of FIG. 24, and FIG. 26 shows depth analysis results of two dark portions (sites denoted by reference numerals 42 and 13 in FIG. 25) assumed to be nano-order depressions in FIG. 25 and surroundings thereof.

As shown in the analysis result shown in FIG. 26, it was found that, among these two depressions, a depression was 0.261 μm (261 nm) in inner diameter, 0.670 μm in depth, and another depression was 0.382 μm (382 nm) in inner diameter and 0.370 μm in depth.

Assuming that there is the collision of the water droplets having the particle diameter of about ⅓ of the inner diameter of the depressions from these sizes of the depressions, a depression is considered to be a collision mark of the water droplets of about 80 nm to 90 nm, and the other depression is considered to be a collision mark of the water droplets of about 120 nm to 130 nm.

Therefore, it is considered that a large number of collision marks due to collision of the water droplets of about 80 nm to 130 nm are present in the sample sprayed with the high-speed nano mist.

Therefore, it can be estimated that a large number of water droplets having the particle diameter of about 80 nm to 130 nm are contained in the high-speed nano mist used in the test. It is said that the liquid droplet of a water molecule has the particle diameter of about 0.38 nm, and thus within the above range, an aggregate of about several hundreds of water molecules is considered to be a main component.

FIG. 27 shows a state after oxygen at the gauge pressure of 4 atm (absolute pressure: 5 atm) was sprayed for 5 sec onto a biofilm composed of Staphylococcus aureus attached onto an artificial blood vessel. FIG. 27 is a photograph (SEM: 10 kV, 2000 times) taken with a scanning electron microscope.

The state shown in FIG. 27 is almost the same as before oxygen is sprayed, and the biofilm is not removed at all by oxygen spraying. It is known that this type of biofilm cannot be easily removed, and it is said that the biofilm cannot be removed even though the biofilm is immersed in a chemical for about 24 hours.

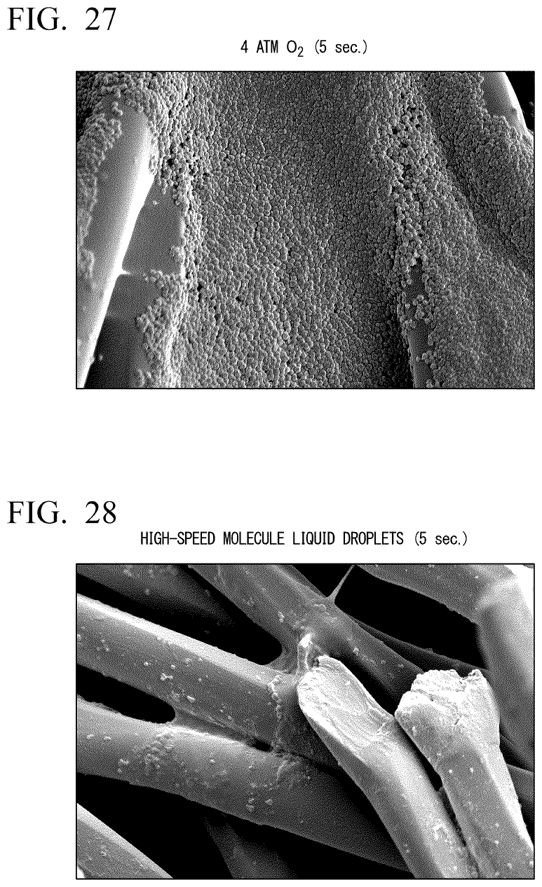

FIG. 28 is an electron microscope photograph (SEM: 10 kV, 2000 times) showing a state after ejecting, for 5 sec from the jet nozzle, the high-speed nano mist of the water generated by evaporating the water in the sealed container while supplying air of 4 atm into the sealed container at a position separated by 4 cm from a biofilm equivalent to the biofilm shown in FIG. 27.

As shown in FIG. 28, the biofilm attached around the artificial blood vessel was almost completely removed as a result of ejecting high-speed nano mist for 5 sec. As shown in FIG. 27, the biofilm was hardly removed by spraying oxygen, but the biofilm was removed in just 5 sec by spraying the high-speed nano mist onto the biofilm.

The portion from which the biofilm is removed is not wet at all, and thus it is possible to clean and sterilize the biofilm in a dry state. The high-speed nano mist quickly vaporizes after colliding with the corresponding portion, and a next high-speed nano mist also sequentially vaporizes after colliding with the corresponding portion. Therefore, as a result, the site to which the high-speed nano mist is sprayed is cleaned and sterilized without being wet.

From the comparison described above, it is clear that the biofilm can be removed in a short time by spraying the high-speed nano mist, and the cleaning is completed in the dry state. Therefore, dry sterilization can be easily performed on the site at which the biofilm is generated.

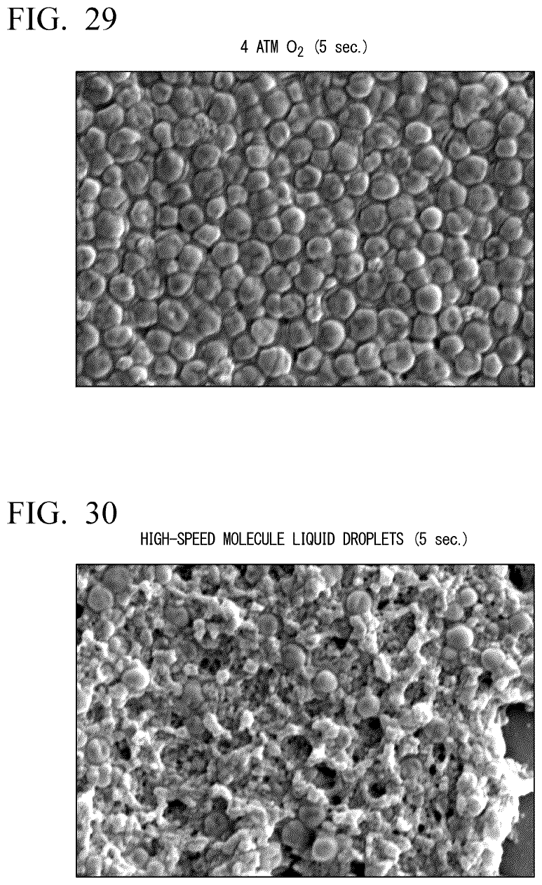

FIG. 29 is a microscope photograph (SEM: 10 kV, 9000 times) showing a state after oxygen at the gauge pressure of 4 atm (absolute pressure: 5 atm) is sprayed for 5 sec to the biofilm composed of Staphylococcus aureus formed on a stainless steel substrate.

It is clear that the state shown in FIG. 29 is almost the same as before oxygen is sprayed, and that the biofilm generated on the stainless steel substrate cannot be removed by spraying oxygen.

FIG. 30 is a microscope photograph (SEM: 10 kV, 9000 times) showing a state after ejecting, for 5 sec from the jet nozzle, the high-speed nano mist of the water generated by evaporating the water in the sealed container while supplying air at the gauge pressure of 4 atm to the sealed container is sprayed at a position separated by 4 cm from a biofilm equivalent to the biofilm shown in FIG. 29.

As shown in FIG. 30, it can be seen that most of Staphylococcus aureus present on a surface side of the biofilm has been destroyed and removed. When the high-speed nano mist is further sprayed for a longer time from the state shown in FIG. 30, the biofilm was almost completely removed.

Therefore, it is possible to attain a cleaning effect and a sterilization effect by ejecting the high-speed nano mist for a site at which propagation of Staphylococcus aureus is concerned or a site at which propagation of other bacterium is concerned. The portion from which the biofilm is removed is not wet at all, and thus it is possible to clean and sterilize the film in a dry state.

These sites at which the cleaning effect and the sterilization effect can be attained are not limited to a part of the human body such as the artificial blood vessel described above, and may be a surface of the stainless steel substrate. Therefore, it can be assumed that the cleaning effect and the sterilization effect can be attained for hand cleaning, dry shower, dry sterilizing of an instrument and the like, dry sterilizing for food, and cleaning of the substrate and the like, as described above.

Based on the analysis of the results shown in FIGS. 29 and 30, the following can be estimated.

Staphylococcus aureus has a hard cell wall containing a peptidoglycan as a main component, and has a so-called balloon structure containing a substance softer than the cell wall of chromosomal DNA, ribosome, mitochondria, and the like on an inner side of the cell wall. It can be estimated that, by spraying the high-speed nano mist, the high-speed nano mist destroys the cell wall of Staphylococcus aureus, applies, for example, an action of rupturing the balloon with a bullet or a needle, and destroys Staphylococcus aureus one by one.

By analyzing the phenomenon, it is considered that, for example, when the high-speed nano mist is sprayed against the virus or the bacterium floating in the air, the cell membrane of the bacterium in the air can be destroyed or damaged, and the cell can be killed or inactivated. When the virus floats in the air, a lipid bilayer membrane constituting an outer layer of the virus may be destroyed or damaged, and the virus may be destroyed or inactivated. Alternatively, by dropping the virus downward with the high-speed nano mist, the virus floating in the air can be inactivated so as not to be absorbed by the human body.

Therefore, it is considered that the cleaning and the sterilization of the space can be performed by generating the mist curtain by the high-speed nano mist by spraying the high-speed nano virus to the space of a site in which the sterilization and the cleaning are necessary. Therefore, as described above, it is considered that the mist curtain of the high-speed nano mist can be implemented by ejecting the high-speed nano mist into the space instead of the acrylic plate used for current virus protection, and an effect of the virus protection can be exhibited.

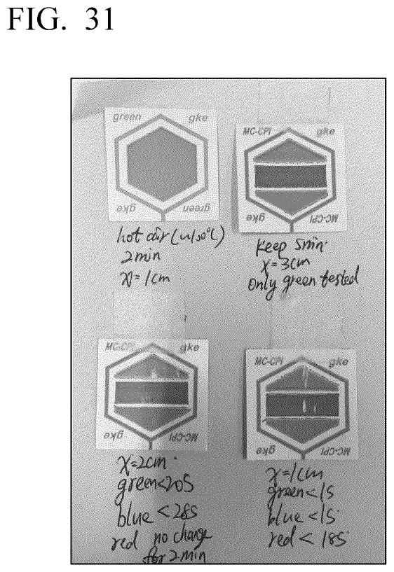

FIG. 31 is a photograph showing a result of a cleaning test performed to check the cleaning effect by the high-speed nano mist.

In the cleaning test, a gke cleaning process monitoring indicator manufactured by a gke-GmbH company (Germany) and imported and sold by Meiyu Co., Ltd. (Japan) was used.

The monitoring indicator is a monitoring indicator obtained by combining a plurality of test papers obtained by printing, for each color, a print mark of a shape filled in a regular hexagonal shape displayed at the upper left corner of the photograph in FIG. 31. In the cleaning test, a test paper in which the print mark is formed in yellow, a test paper in which the print mark is formed in blue, a test paper in which the print mark is formed in green, and a test paper in which the print mark is formed in red are used. A yellow test paper, a blue test paper, a green test paper, and a red test paper are printed in this order such that coating films of the print marks are sequentially hardened.

The regular hexagonal shape print mark displayed at the upper left of the photograph in FIG. 31 is the test paper on which a green print mark is printed. In a print paper, as in a case of the print mark displayed on the upper right of FIG. 31, there is also a test paper in which a regular hexagonal region was divided into three regions of a green region, a blue region, and a red region in this order from the top, and a cleaning test was performed by appropriately using these test papers.

First, an irradiation distance from the distal end of the jet nozzle was fixed to 1 cm to 4 cm, irradiation time was set to 1 sec or 5 sec, and a comparative cleaning test was performed with respect to a case in which only heated air was irradiated (heated air temperature: 30° C., distance between the injection nozzle and the test paper: 1 cm, injection speed: 20 m/s, irradiation: 2 minutes).

When only the heated air was irradiated, no discoloration was detected when the test paper having the yellow print mark was used, and detergency was not confirmed.

However, when the irradiation distance is 4 cm, the discoloration of any color print mark was not confirmed, whereas when the irradiation distance is 3 cm, slight discoloration of only the yellow and green print marks was confirmed.

Similarly, when the irradiation distance is 2 cm or 1 cm, the slight discoloration of only the green print mark was confirmed.

As indicated by the test paper displayed on the upper right of the photograph in FIG. 31, when a test was performed in which the irradiation distance was set to 3 cm at the gauge pressure of 4 atm (absolute pressure: 5 atm) and only the green region printed at the uppermost position was ejected with the high-speed nano mist, no discoloration occurred in the green region.

As indicated by the test paper displayed on the lower left of the photograph in FIG. 31, when the irradiation distance was fixed to 2 cm at the gauge pressure of 4 atm (absolute pressure: 5 atm) and the green region printed at the uppermost position was irradiated for 20 sec, clear discoloration occurred, and thus it was confirmed that the detergency was attained.

When the irradiation distance was fixed to 2 cm and the blue region located at a center was irradiated for 20 sec at a gauge pressure of 4 atm (absolute pressure: 5 atm), the clear discoloration occurred, and thus it was confirmed that the detergency was attained. In the cleaning test, since the red region located at a lowest position was not irradiated, no change was observed in the red region.

As indicated by the test paper displayed on the lower right of the photograph in FIG. 31, when the irradiation distance was fixed to 1 cm at the gauge pressure of 4 atm (absolute pressure: 5 atm) and the green region printed at the uppermost position was irradiated for 1 sec, the clear discoloration occurred, and thus it was confirmed that the detergency was attained.

When the irradiation distance was fixed to 1 cm at the gauge pressure of 4 atm (absolute pressure: 5 atm) and the blue region located at the center was irradiated for 1 sec, the discoloration clearly occurred, and thus it could be confirmed that the detergency was implemented.

When the irradiation distance was fixed to 1 cm at the gauge pressure of 4 atm (absolute pressure: 5 atm) and the red region located at the lowest position was irradiated for 18 sec, no discoloration occurred, and thus it was confirmed that the detergency for cleaning a paint in the red region was not attained.

As described above, by spraying the high-speed nano mist onto the print mark of each test paper, magnitude of the detergency of the high-speed nano mist according to the first embodiment was confirmed.

### Example 2

The sealed container 6 having the structure shown in FIG. 15 was prepared. The bottom plate 11, the top plate 12B, and the strut member 15 were formed of SUS 316 specified by JIS. The bottom plate 11 having an outer diameter of 110 mm and a thickness of 12 mm and the top plate 12B having an outer diameter of 110 mm and a thickness of 15 mm are prepared, the wall body 13 is implemented by a cylindrical body made of quartz glass, and the bottom plate 11, the top plate 12B, and the wall body 13 were combined to constitute the cylindrical sealed container 6 having a total height of 150 mm. The jet nozzle is made of SUS 316 specified by JIS. A circular concave portion having a depth of 7 mm was formed on an upper surface side of the bottom plate 11 and a lower surface side of the top plate 12B, a bottom portion and a top portion of the wall body 13 were fitted into the concave portion via an O-ring, the strut members were aligned with counterbore portions of the bottom plate 11 and the top plate 12, and the strut members were bolted and assembled in a cylinder shape, whereby the sealed container 6 was assembled. In the jet nozzle 8, the tubular portion 8A had a diameter of φ8 mm, and the jet nozzle 8 is used in which a water path having a diameter of φ4.5 mm was formed in the tubular portion 8A and the nozzle hole 8D having a diameter of φ0.7 mm was formed in a central portion of a distal end wall B. The size of the sealed container described above is a size that does not require registration of the pressure container, and is merely used as an example.

The built-in heater 3B was provided inside the sealed container 6. The gas supply pipe 9B was attached to the periphery of the wall body 13 of the sealed container 6, connected to the gas supply source 2 implemented by a gas cylinder, the temperature sensor 23 (E5CN-HQ2 manufactured by Omron, KTO-16150M3 manufactured by AS ONE Corporation) was connected to the sealed container 6, the sealing nut 20 was removed from the joint member 19, and 200 mL of water was injected into the sealed container from an inlet of the joint member 19. Similarly, the temperature sensor 23B was provided near the nozzle. The water is injected such that a residual space having a height of about 2 cm was left in the sealed container 6.

After the water is injected, the sealing nut 20 was closed to seal the sealed container 6. Thereafter, water was heated by the built-in heater 3B, and the jet pipe 7 was heated to a temperature equal to or higher than the boiling point of the water by a heater (ribbon heater R1111, manufactured by Tokyo Technological Labo). Similarly, the top plate 12 and the gas supply pipe 9 were heated to the temperature equal to or higher than the boiling point of the water by the heater 65. Air was supplied from the gas supply source 2 to the residual space of the sealed container 6, an air pressure in the residual space was gradually increased at regular intervals to adjust the gauge pressure to 1 atm to 4.8 atm (2 atm to 5.8 atm as the absolute pressure in the sealed container), and the sealed container 6 was heated by the built-in heater 3B to heat the water in the sealed container to the boiling temperature. Specifically, a set temperature of the built-in heater was set to about 152° C. The pressure in the sealed container was confirmed by a pressure gauge.

Condensed water may be generated by condensing the high-speed nano mist in the jet pipe 7. A generation frequency of the condensed water during the production of the high-speed nano mist in the nano mist production device in FIG. 15 was measured. For the measurement, a laser source (SDL-532-100TL manufactured by Shanghai Dream Laser technology), a photoelectric converter, and an oscilloscope (WaveSurfer 510 manufactured by Teledyne LeCroy, sample rate 400 μs) were used. The laser, the photoelectric converter, and the jet nozzle 8 were disposed at the same height and measured. A change in laser intensity is read by the photoelectric converter and recorded in the oscilloscope. Each time the condensed water passes through laser light, the laser light is blocked, and a great change in the voltage occurs. By measuring the great change in the voltage, the number of times of generation of the condensed water can be measured. FIG. 32 shows the voltage change during the high-speed nano mist production performed by the nano mist production device shown in FIG. 1. In FIG. 32, a horizontal axis indicates time (min), and a vertical axis indicates the voltage change. In FIG. 32, a plurality of peaks appear, and this indicates that the condensed water passes. From FIG. 32, it was found that, when the nozzle was not heated, the condensed water was generated at a high frequency.

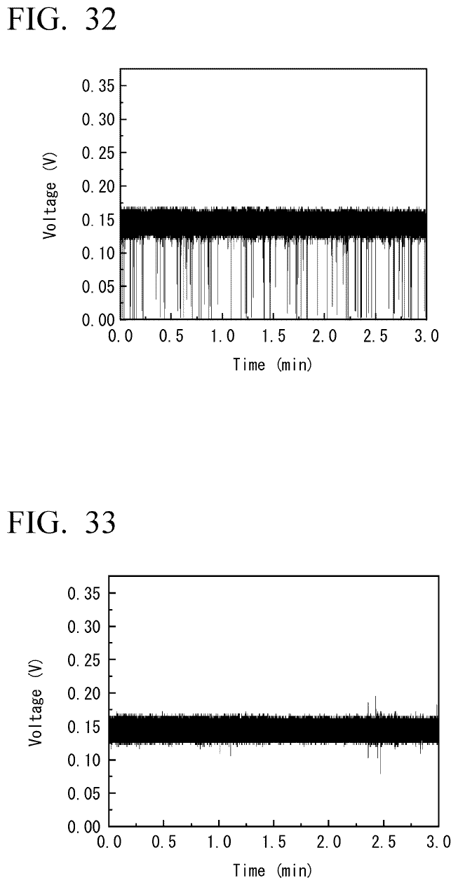

FIG. 33 shows a voltage change when the nano mist is produced by heating the jet nozzle to 180° C. by the nano mist production device in FIG. 15. In FIG. 33, a horizontal axis indicates time (min), and a vertical axis indicates the voltage change. As is clear from FIG. 33, it was found that the number of times of generation of the condensed water was reduced using the nano mist production device in FIG. 15 by heating the entire nano mist production device.

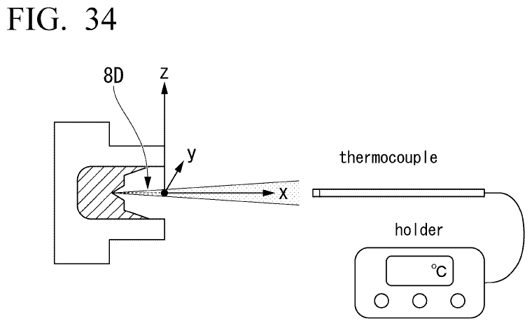

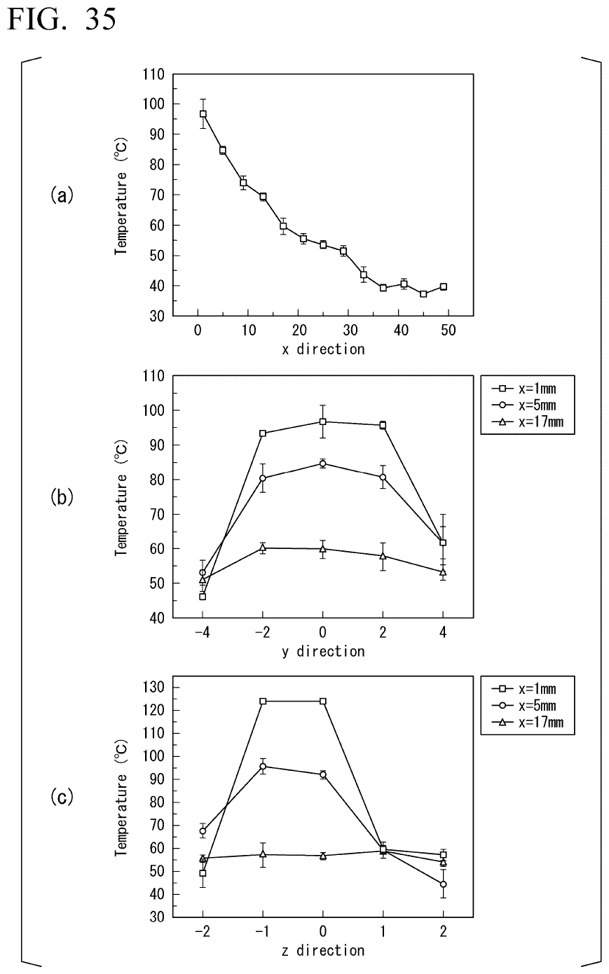

The nano mist production device in FIG. 15 has a size of liquid droplets in the mist smaller than that of the nano mist production device in FIG. 1, and thus it is difficult to visualize the mist by a high-speed camera. Therefore, a macro feature of the high-speed nano mist was measured. FIG. 34 is a diagram showing arrangement of a measurement device that measures temperature distribution of the high-speed nano mist. An extending direction of the jet nozzle 8 is an x axis, an axis orthogonal to the x axis is a y axis, and an axis orthogonal to the x axis and the y axis is a z axis. A position that is a center of the nozzle hole 8D in a YZ plane and that is the distal end of the jet nozzle 8 at the x axis is an origin. A pressure was set to 5 atm, the high-speed nano mist was produced, and a temperature at each position was measured by a thermocouple. The temperature distribution varies depending on a shape of the nozzle. The distribution in (a) of FIG. 35, (b) of FIG. 35, and (c) of FIG. 35 is an example of the temperature distribution. (a) of FIG. 35 shows the temperature distribution in the x axis direction (y=0 mm, and z=0 mm). In (a) of FIG. 35, a horizontal axis indicates an x direction (mm), and a vertical axis indicates the temperature (° C.). As shown in (a) of FIG. 35, as the distance from the jet nozzle 8 increases, the temperature rapidly decreases, and the temperature is relatively stable from 35 mm to 49 mm on the x axis. (b) of FIG. 35 shows the temperature distribution in a y-axis direction (z=0), and (c) of FIG. 35 shows the temperature distribution in a z-axis direction (y=0). The temperature distribution on the y axis and the z axis is measured by changing a position of an x coordinate. In (b) of FIG. 35, a horizontal axis indicates a y direction (mm), and a vertical axis indicates the temperature (° C.). In (c) of FIG. 35, a horizontal axis indicates a z direction (mm), and a vertical axis indicates the temperature (° C.). As shown in (b) of FIG. 35, a temperature change in the y-axis direction was symmetrical about the origin, and a temperature change in the z-axis direction was changed symmetrically with respect to the position moved in a negative direction from the origin.

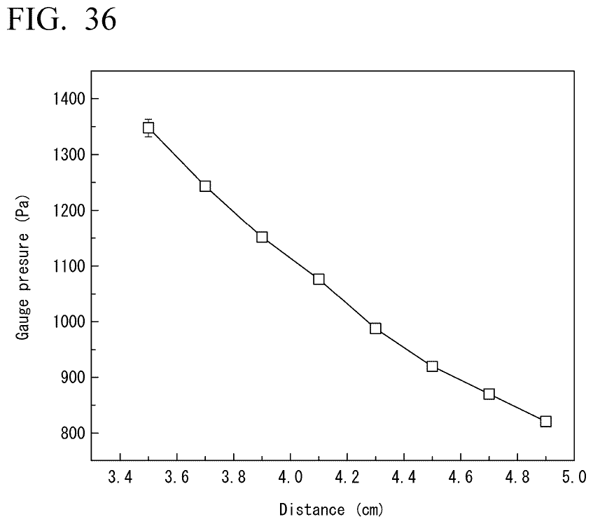

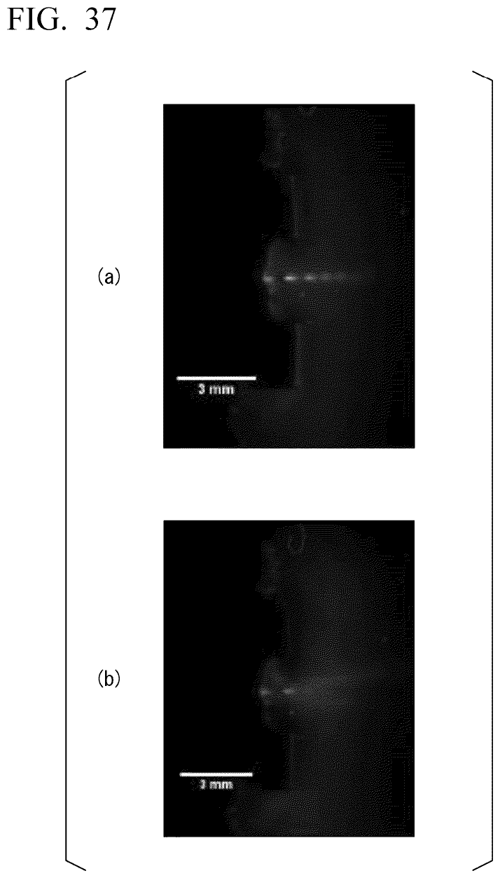

Next, the pressure distribution of the mist was measured. The pressure distribution was measured using a pitot tube (LK-00 manufactured by Okano Works, Ltd.) and a flow meter (FV-21 manufactured by Okano Works, Ltd.). The pressure distribution was measured in a range from a position of 3.5 cm to a position of 4.9 cm from the jet nozzle 8. FIG. 36 shows a relation between a total pressure and the position that are obtained by the measurement. In FIG. 36, a horizontal axis indicates the position (mm), and a vertical axis indicates the total pressure (Pa). As shown in FIG. 36, the total pressure decreased as the distance increases.

The nano mist production device in FIG. 15 was visualized by a schlieren method. As a light source, a xenon lamp (LS-300 manufactured by Kato Koken) was used. The nano mist was produced at 5 atm. The jet nozzle was disposed such that the high-speed nano mist flows perpendicularly to the light. An obtained result is shown in FIG. 37. (a) of FIG. 37 shows a schlieren image of the gas flow (in a case of only gas) before heating, and (b) of FIG. 37 shows a schlieren image of the nano mist (water vapor mixed gas) after heating. As shown in (a) of FIG. 37, the gas that is ejected from the jet nozzle has exceeded a sonic speed. Similarly, it was found that the high-speed nano mist also exceeded the sonic speed immediately after being ejected from the nozzle. A supersonic speed region of the high-speed nano mist was reduced as compared with the case of the gas. It is considered that the above is because the speed was lowered due to the condensation of the high-speed nano mist.

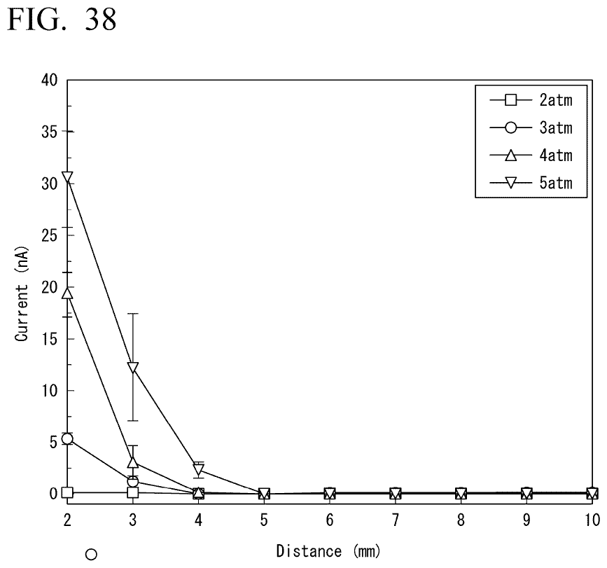

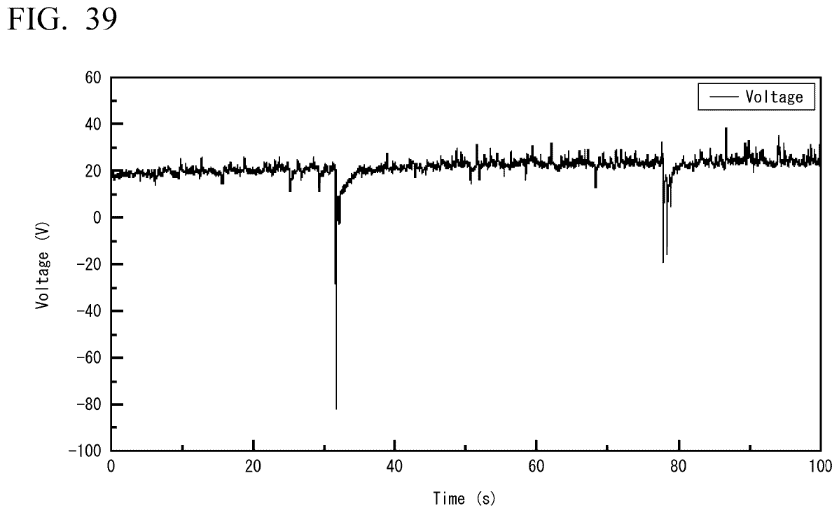

Next, similarly to an example 1, the aluminum plate was irradiated with the high-speed nano mist, and the flowing current was measured. FIG. 38 shows a relation between a current that flows when the aluminum plate is irradiated with the high-speed nano mist and the separation distance between the jet nozzle and the aluminum plate. In FIG. 38, a horizontal axis indicates the distance (mm) between the jet nozzle and the aluminum plate, and a vertical axis indicates the current (nA). As shown in FIG. 38, the higher the pressure and the smaller the distance was, the more the current flowed. However, as compared with the nano mist production device in FIG. 1, the flowing current became smaller. It is considered that the above is because the size of the liquid droplets was smaller than that in the example 1. It is considered that the above is because the time until evaporation of the small liquid droplets becomes short, and thus the liquid droplets do not fly that long.

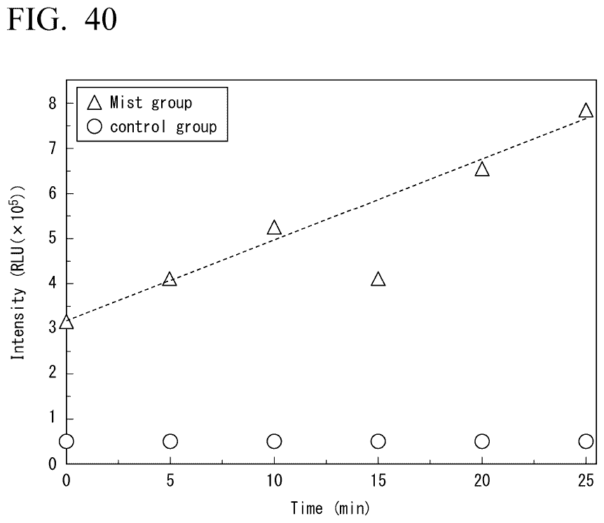

Next, a potential of the aluminum plate was measured using an electrostatic voltmeter (244A manufactured by Monoe Electronics). FIG. 39 shows a relation between the potential of the aluminum plate and the time when the high-speed nano mist is irradiated at the distance of 2 mm between the jet nozzle and the aluminum plate and the pressure of the absolute pressure of 5 atm (gauge pressure: 4 atm). It is considered that a value of a peak appearing in FIG. 39 is caused by relatively large liquid droplets, and that an average potential is caused by the nano mist of less than 1 μm. A method for measuring the state of the ejected mist can be used.

An amount of hydrogen peroxide in the high-speed nano mist produced by the nano mist production device in FIG. 15 was measured. In the measurement, a luminometer (Luminescencer PSN AB 2200/AB-2200R manufactured by ATTO) was used. The measurement was performed by condensing and collecting the high-speed nano mist. The sample was collected every 5 minutes. The amount of hydrogen peroxide was evaluated by reacting a luminol reaction reagent manufactured by Fuji Film Co., Ltd. with hydrogen peroxide in the sample, and detecting light at the time of reaction. Ultrapure water was also measured for comparison. An obtained result is shown in FIG. 40. In FIG. 40, a horizontal axis indicates the time, and a vertical axis indicates an intensity of light. The intensity of light on the vertical axis correlates with a concentration of the hydrogen peroxide due to the intensity of light that is emitted by reacting with hydrogen peroxide. A hydrogen peroxide solution in the ultrapure water was hardly detected. On the other hand, the intensity of the high-speed nano mist increased with lapse of the time. The above indicates that the hydrogen peroxide solution was produced in the high-speed nano mist. From the above, it was confirmed that hydrogen peroxide was also produced with the high-speed nano mist.

### REFERENCE SIGNS LIST

A: nano mist production device
M: high-speed nano mist
1: nano mist production device main body
2: gas supply source
3: heating device
4: temperature measuring device
6: sealed container
7: jet pipe
8: jet nozzle
8D: nozzle hole
10: nozzle heater
11: bottom plate
12: top plate
13: wall body
15: strut member
23: temperature sensor
30: hand (object)
31: human body (object)
36: cooking tool (object)
37: human body (object)
38: foodstuff (object)
39: substrate (object)
41: cow (object)

1. A high-speed nano mist being a group of liquid droplets having a particle diameter of 1 nm to 10000 nm and flying at a speed of 50 m/s to 1000 m/s.

2. A production method for a high-speed nano mist for producing the high-speed nano mist which is a group of liquid droplets having a particle diameter of 1 nm to 10000 nm and flying at a speed of 50 m/s to 1000 m/s.

3. The production method for a high-speed nano mist according to claim 2, the production method comprising
using water as the high-speed nano mist, and ejecting water vapor from the water contained in a sealed container and a pressurized gas supplied to the sealed container from a jet nozzle provided in the sealed container.

4. A processing method comprising
performing at least one of sterilization, cleaning, and surface processing in a state in which a usage amount of a liquid is reduced without using a drug in a dried state by generating a high-speed nano mist, which is a group of liquid droplets having a particle diameter of 1 nm to 10000 nm and flying at a speed of 50 m/s to 1000 m/s, and by causing the high-speed nano mist to collide with a target object.

5. The processing method according to claim 4, further comprising
using water as the high-speed nano mist, and ejecting water vapor from the water contained in a sealed container and a pressurized gas supplied to the sealed container from a jet nozzle provided in the sealed container.

6. The processing method according to claim 4, wherein
a phenomenon is used in which OH radical or hydrogen peroxide is generated at a time of producing the high-speed nano mist.

7. A measurement method for a high-speed nano mist, wherein
a phenomenon in which a current flows or a phenomenon in which a voltage changes at a collision surface of a conductor to which the high-speed nano mist is sprayed is used by producing the high-speed nano mist and spraying the high-speed nano mist to the conductor, the high-speed nano mist being a group of liquid droplets having a particle diameter of 1 nm to 10000 nm and flying at a speed of 50 m/s to 1000 m/s.

8. A production device for a high-speed nano mist for producing the high-speed nano mist, which is a group of liquid droplets having a particle diameter of 1 nm to 10000 nm and flying at a speed of 50 m/s to 1000 m/s, and causing the high-speed nano mist to collide with a target object.

9. The production device for a high-speed nano mist according to claim 8, the production device comprising:
a sealed container configured to use water as the high-speed nano mist and to contain the water;
a gas supply source configured to supply a pressurized gas to the sealed container; and
a jet nozzle configured to eject water vapor from the water and the pressurized gas supplied to the sealed container.

10. A processing device for performing at least one of sterilization, cleaning, and surface processing in a state in which a usage amount of a liquid is reduced without using a drug in a dried state by producing a high-speed nano mist, which is a group of liquid droplets having a particle diameter of 1 nm to 10000 nm and flying at a speed of m/s to 1000 m/s, and by causing the high-speed nano mist to collide with a target object.

11. The processing device according to claim 10 comprising:
a sealed container configured to use water as the high-speed nano mist and to contain the water;
a gas supply source configured to supply a pressurized gas to the sealed container; and
a jet nozzle configured to eject water vapor from the water and the pressurized gas supplied to the sealed container.

12. A measurement device for a high-speed nano mist for measuring a current flowing or a voltage generated on a collision surface of a conductor to which the high-speed nano mist is sprayed by producing the high-speed nano mist and spraying the high-speed nano mist to the conductor, the high-speed nano mist being a group of liquid droplets having a particle diameter of 1 nm to 10000 nm and flying at a speed of 50 m/s to 1000 m/s.
